(19) United States
(12) Patent Application Publication
(10) Pub. No.: US 2025/0057917 A1
(43) Pub. Date: Feb. 20, 2025

(54) COMPOSITION AND METHODS FOR TREATING RADIATION INDUCED INJURY

(71) Applicant: Xequel Bio, Inc., Fort Worth, TX (US)

(72) Inventors: Carissa HAYNES, Mt. Pleasant, SC (US); Christina L. GREK, Mt. Pleasant, SC (US)

(21) Appl. No.: 18/800,237

(22) Filed: Aug. 12, 2024

Related U.S. Application Data
(60) Provisional application No. 63/519,425, filed on Aug. 14, 2023.

(51) Int. Cl. A61K 38/17; A61K 9/06; A61K 47/38; A61P 17/02

(52) U.S. Cl. CPC A61K 38/1709; A61K 9/06; A61K 47/38; A61P 17/02

(57) ABSTRACT

The present disclosure provides compositions and methods for use in treating radiation injury such as a radiation induced skin injury in a subject at risk of such injury. Also provided herein is a reproducible animal model of cutaneous radiation injuries for evaluation of medical countermeasures.

### CROSS REFERENCE TO RELATED APPLICATIONS

This reference claims the benefit of priority of U.S. Provisional Application No. 63/519,425, filed on Aug. 14, 2023, the entire contents of which are hereby incorporated by reference.

### REFERENCE TO AN ELECTRONIC SEQUENCE LISTING

The contents of the electronic sequence listing (FIRS_015_01US_SeqList_ST26.xml; Size: 82,838 bytes; and Date of Creation: Aug. 9, 2024) is herein incorporated by reference in its entirety.

### BACKGROUND

A nuclear or radiological incident could lead to exposure of civilians to high doses of radioactive material. In addition to acute radiation syndrome (ARS), exposure to ionizing radiation can severely damage the skin, causing cutaneous radiation injuries (CRI). CRI often results in severe skin tissue ulceration and necrosis, requiring invasive surgical interventions such as wound excision and grafting or amputation. Animal models that respond to radiation in a manner predictive of humans is a necessity for development of medical countermeasures (MCMs). There is no available animal model that recapitulates the complex dermal wounds associated with severe, full-thickness CRI resulting from ionizing radiation exposure.

The methods and compositions provided herein address this unmet need.

### SUMMARY

In one aspect, provided herein is a method of treating a radiation induced skin injury on a subject, comprising: (a) debriding a radiation induced skin injury on a subject exposed to a source of radiation; and (b) administering to the subject a composition comprising at least one alpha connexin polypeptide in a dosing regimen effective for the treatment of the radiation induced skin injury, and wherein step (a) is repeated at defined intervals during the dosing regimen effective for the treatment of the radiation induced skin injury. In some cases, the dosing regimen effective for the treatment of the radiation induced skin injury is daily for a plurality of days. In some cases, the plurality of days is 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115 or 120 days. In some cases, the defined intervals is daily, every 2 days, every 3 days, every 4 days, every 5 days or every 6 days. In some cases, the composition is administered to the subject at a dose of from about 10 μM to about 2000 μM. In some cases, the composition is administered to the subject at a dose of from about 100 μM to about 200 μM. In some cases, the composition is administered to the subject at a dose of about 1 mg/kg to about 50 mg/kg. In some cases, the composition is administered topically. In some cases, the composition further comprises hydroxyethylcellulose gel. In some cases, the hydroxyethylcellulose gel is present at a concentration of about 1.25% (w/w). In some cases, the at least one alpha connexin polypeptide comprises the carboxy terminal-most 4 to 30 contiguous amino acids of an alpha Connexin, or a conservative variant thereof. In some cases, the alpha connexin polypeptide is connexin 37, connexin 40, connexin 43, or connexin 45. In some cases, the at least one alpha connexin polypeptide is linked at its amino terminus to a cellular internalization transporter. In some cases, the cellular internalization transporter is an antennapedia sequence. In some cases, the at least alpha connexin polypeptide comprises the amino acid sequence of SEQ ID NO: 9. In some cases, the at least alpha connexin polypeptide comprises the amino acid sequence of SEQ ID NO: 91. In some cases, step (b) is performed after the onset of erythema, desquamation, ulceration or any combination thereof. In some cases, step (b) is performed after the onset of erythema. In some cases, the radiation induced skin injury is selected from the group consisting of cutaneous radiation injury (CRI), radiation burns, and radiation dermatitis. In some cases, the source of radiation is selected from the group consisting of a medical intervention, a diagnostic machine, the sun, a tanning bed, weapons of mass destruction (WMD), nuclear explosions, and dirty bombs. In some cases, the method reduces the time to 50% wound closure of the radiation induced skin injury as compared to standard of care treatment for wound healing of the radiation induced skin injury. In some cases, the method reduces the time to 100% wound closure of the radiation induced skin injury as compared to standard of care treatment for wound healing of the radiation induced skin injury. In some cases, the method increases the incidence or frequency of 100% complete wound closure of the radiation induced skin injury as compared to standard of care treatment for wound healing of the radiation induced skin injury. In some cases, the method increases the average percent of wound closure of the radiation induced skin injury as compared to standard of care treatment for wound healing of the radiation induced skin injury. In some cases, the method accelerates wound re-epithelialization of the radiation induced skin injury by at least 5%, 10%, 15%, 20%, 25%, 30%, 35%, 40%, 45%, 50%, 55%, 60%, 65%, 70%, 75%, 80%, 85%, 90%, 95% 99% or 100% as compared to standard of care treatment for wound healing of the radiation induced skin injury. In some cases, the debriding performed in step (a) comprises selective debridement, non-selective debridement or a combination thereof. In some cases, the debriding performed in step (a) comprises one or more types of debridement selected from the group consisting of autolytic, surgical, mechanical, irrigation, biological, enzymatic and any combination thereof. In some cases, the type of debridement performed in step (a) is determined following evaluation by a caregiver. In some cases, the caregiver is a certified medical professional.

### BRIEF DESCRIPTION OF THE FIGURES

FIG. 1 depicts signs of radiation injuries caused by external exposure to high doses of ionizing radiation.

FIG. 2 describes aspects of the FDA's animal rule around animal models developed as medical countermeasures (MCMs) against Chemical, Biological, Radiological, and Nuclear (CBRN) threats.

FIG. 3 illustrates a general overview of the timeline for use of the Yorkshire swine model for inducing and treating cutaneous radiation injury (CRI) as described in Example 1.

FIG. 4A-4C depicts the radiation physics associated with the Yorkshire swine model. FIG. 4A depicts percent depth dose (PDD) measurement Left: Setup. 25×25 cm2 electron cone with Cerrobend cutout and lead skin collimator. Eight mm Superflab bolus, films placed at 8 mm and 12 mm depth, and solid water slabs for backscatter on top of the Advanced Markus® chamber. Right: PDD curve was quantified by converting the measured percent depth ionization curve with corrections using the stopping power ratios at different depths FIG. 4B depicts output factor and dose profiles measured by EBT-XD Gafchromic™ film. Top: Film pieces irradiated at 8-and 12-mm depths. Bottom: Measured dose profiles at 8-and 12-mm depths. FIG. 4C depicts end-to-end test with a pig-sized water phantom. Left: A cylindrical water phantom irradiation setup with four irradiation sites aligned along the LINAC y-axis (head-to-toe) and positioned for irradiation. Right: Measured dose profiles from films for four irradiated sites at 8 mm depth.

FIG. 5 depicts the irradiation steps utilized in the Yorkshire swine CRI model.

FIG. 6 depicts supportive care procedures including debridement, pain management and infection management protocols utilized in the Yorkshire swine CRI model.

FIG. 7A-7B illustrates how the Yorkshire Swine Model reproduces the natural history of cutaneous radiation injury (CRI) in bumans. FIG. 7A shows the percent of irradiated sites presenting with skin phenotypes during daily clinical evaluations. Irradiated sites follow progression observed in documented incidences of CRI in humans; a latency period is followed by erythema, desquamation, ulceration, and necrosis within the 120-day study period. FIG. 7B shows the linear regression of individual irradiated site wound size across the study period. Optimization of the Yorkshire swine model of CRI across studies improves reproducibility.

FIG. 8A depicts representative images from irradiated skin sites in Yorkshire swine model. Ruler is used for planimetric measurements. FIG. 8B depicts mean ulcer area by treatment group from post-irradiation Day 70 to Day 120 (necropsy) measured using planimetry performed on wound photographs. FIG. 8C depicts incidence of closed irradiation wounds by treatment group from post-irradiation Day 70 to Day 120 (necropsy) from daily clinical assessments of wound status. *p<0.05, **p<0.01, linear mixed effects model; n=32 CRI wounds/treatment arm.

FIG. 9A depicts in vitro cell proliferation assay demonstrating an increase in keratinocyte proliferation with aCT1 (active pharmaceutical ingredient in Granexin) treatment. Student's t-test, *p<0.0001; n=8; mean±SEM. FIG. 9B depicts in vitro cell migration assay demonstrating an increase in keratinocyte migration with aCT1 treatment. Student's t-test, *p<0.0018; n=3; mean±SEM. FIG. 9C depicts proposed mechanism of action of Granexin in promoting wound healing.

### DETAILED DESCRIPTION

### Definitions

While the following terms are believed to be well understood by one of ordinary skill in the art, the following definitions are set forth to facilitate explanation of the presently disclosed subject matter.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

As used herein, the term “a” or “an” can refer to one or more of that entity, i.e. can refer to a plural referents. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

As used herein, the term “selective debridement” can refer to debriding that targets devitalized tissue only, while the term “non-selective debridement” as used herein, can refer to debriding that targets the entire wound, including healthy tissue. Examples of “selective debridement” as used herein can be surgical debridement, enzymatic debridement, autolytic debridement and biological debridement. Examples of “non-selective debridement” as used herein can be mechanical debridement and irrigation debridement.

The terms “surgical debridement”, “operative debridement”, “excisional debridement” and “sharp debridement” can be used interchangeably and can refer to the removal of tissue with forceps, scalpels or medical scissors.

As used herein, the term “enzymatic debridement” can refer to any debriding that utilizes chemical or enzymatic agents to break down necrotic tissue.

As used herein, the term “autolytic debridement” can refer to any debriding that utilizes a subject's own defense mechanisms and fluids to liquefy eschar, slough, and other forms of necrotic tissue.

As used herein, the term “mechanical debridement” can refer to any debriding that utilizes dressings such as, for example, wet-to-dry dressings or wet gauze dressings that can pull necrotic debris out of the wound and transfer it into the dressing.

As used herein, the term “biological debridement” can refer to any debriding that utilizes one or more organisms to facilitate debridement of a wound in a subject.

As used herein, the term “irrigation debridement” can refer to any debriding that utilizes fluid to remove wound debris topical agents, and surface bacteria from the wound in a subject

As used herein, “subject” can include, but may not be limited to, animals, plants, bacteria, viruses, parasites and any other organism or entity that has nucleic acid. The subject may be a mammalian or non-mammalian animal. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. In some embodiments, a patient refers to a subject afflicted with a disease or disorder. In some embodiments, a patient population refers to a particular, defined set of subjects having a disease or disorder or at risk of developing a particular disease or disorder. For example, in some embodiments, the present disclosure provides methods for treating, preventing, or mitigating the severity of radiation injury in a patient population comprising soldiers and/or civilians present in an area subject to war and/or terrorism. In some embodiments, the present disclosure provides methods for treating, preventing, or mitigating the severity of radiation injury in a patient population comprising cancer subjects receiving radiation therapy. The term “patient” includes human and veterinary subjects.

By “treat” or “treatment” is meant a method of reducing the effects of a disease or condition. Treatment can also refer to a method of reducing the underlying cause of the disease or condition itself rather than just the symptoms. The treatment can be any reduction from native levels and can be but is not limited to the complete ablation of the disease, condition, or the symptoms of the disease or condition. For example, a disclosed method for promoting wound healing is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

### Overview

Provided herein are methods and compositions for treating a radiation induced injury in a subject exposed to source of radiation. In some cases, the radiation is an ionizing radiation. In some cases, the radiation is a beta particle radiation. In some cases, the radiation induced injury is any radiation induced injury known in the art and/or provided herein. In some cases, the radiation induced injury is any radiation induced injury known in the art and/or provided herein that results in a skin injury. In some cases, the radiation induced injury is any radiation induced injury known in the art and/or provided herein is a cutaneous radiation injury (CRI). In some cases, the method for treating the radiation induced skin injury (e.g., CRI) entails administering a composition comprising at least one alpha connexin polypeptide to the subject. The at least one alpha connexin polypeptide can be any alpha connexin polypeptide provided herein. In some cases, administration of the composition comprising the at least one alpha connexin polypeptide is preceded by debridement of the radiation induced skin injury. In some cases, the method for treating the radiation induced skin injury (e.g., CRI) comprises repeated administration of the composition comprising the at least one alpha connexin polypeptide. In some cases, the method for treating the radiation induced skin injury (e.g., CRI) comprises repeated administration of the composition comprising the at least one alpha connexin polypeptide, wherein cach administration of the composition comprising the at least one alpha connexin polypeptide is preceded by debridement of the radiation induced skin injury. In some cases, the method for treating the radiation induced skin injury (e.g., CRI) comprises repeated administration of the composition comprising the at least one alpha connexin polypeptide, wherein every other administration of the composition comprising the at least one alpha connexin polypeptide is preceded by debridement of the radiation induced skin injury. In some cases, the method for treating the radiation induced skin injury (e.g., CRI) comprises repeated administration of the composition comprising the at least one alpha connexin polypeptide, wherein every third, fourth, fifth or sixth administration of the composition comprising the at least one alpha connexin polypeptide is preceded by debridement of the radiation induced skin injury. In some cases, the method for treating the radiation induced skin injury (e.g., CRI) comprises repeated administration of the composition comprising the at least one alpha connexin polypeptide, wherein administration of the composition comprising the at least one alpha connexin polypeptide is preceded by debridement of the radiation induced skin injury if evaluation of the radiation induced skin injury following a previous administration of the composition comprising the at least one alpha connexin polypeptide reveals symptoms that warrant debridement. The symptoms can include the presence of necrotic tissue. The debridement of the radiation induced skin injury can be any type of debridement provided herein. The debridement can be selected from the group consisting of autolytic debridement, surgical debridement, mechanical debridement, irrigation debridement, biological debridement, enzymatic debridement and any combination thereof.

In some cases, the methods provided herein for treating a radiation induced skin jury (e.g., CRI) in a subject exposed to a source of radiation can result in an acceleration of wound healing as compared to a control. In some cases, the control comprises use of a standard of care treatment that does not comprise a composition comprising at least one alpha connexin polypeptide. In some cases, the methods provided herein for treating a radiation induced skin jury (e.g, CRI) in a subject exposed to a source of radiation that comprises administration of a composition comprising at least one alpha connexin polypeptide that is preceded by debridement of the radiation induced skin injury at any of the intervals provided herein can result in an acceleration of wound healing as compared to a control. In some cases, the control method comprises use of a standard of care treatment that does not comprise a composition comprising at least one alpha connexin polypeptide and/or debridement of the radiation induced skin injury.

In some cases, the composition comprising the at least one alpha connexin polypeptide for use in a method for treating a radiation induced skin injury as provided herein is administered prior to exposure of the subject to a source of radiation. In other embodiments, the composition is administered to the subject after exposure to a source of radiation but before the presence of any symptoms of a radiation induced skin injury. In other embodiments, the composition is administered to the subject after exposure to a source of radiation and after the presence of one or more symptoms of a radiation induced skin injury selected from the group consisting of erythema, desquamation and necrosis. In other embodiments, the composition is administered to the subject after exposure to a source of radiation and after the presence of erythema.

In some embodiments, the composition comprising the at least one alpha connexin polypeptide for use in a method for treating a radiation induced skin injury as provided herein is administered topically to the subject. In some embodiments, the composition is administered topically to the subject at a dose of from about 10 μM to about 2000 μM. In further embodiments, the composition is administered to the subject at a dose of from about 50 μM, about 100 μM, about 150 μM, about 200 μM, about 300 μM, about 400 μM, about 500 μM, about 600 μM, about 700μM, about 800 μM, about 900 μM, or about 1,000 μM. In some embodiments, the composition is administered parenterally to the subject at a dose of from about 1 mg/kg to about 50 mg/kg. In further embodiments, the composition is administered to the subject at a dose of about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In some embodiments, the composition is administered in a daily dosing regimen.

In some embodiments, the radiation induced injury is a radiation induced skin injury. The radiation induced skin injury can be cutaneous radiation injury (CRI), radiation burns or radiation dermatitis. In certain embodiments, the radiation induced injury is CRI. In some embodiments, the radiation induced injury comprises radiation injury in combination with burns, wounds, infection, or blunt trauma. In some embodiments, the radiation induced injury results from exposure to a source of radiation selected from the group consisting of weapons of mass destruction (WMD), nuclear explosions, and dirty bombs. In some embodiments, the radiation induced injury comprises radiation dermatitis, which is a specific type of radiation injury resulting from radiotherapy regimens or other interventional medical procedures. In some embodiments, the radiation induced injury results from exposure to the sun, x-ray and other diagnostic machines, or ionizing radiation-emitting sources such as tanning beds. In some embodiments, the peptides provided herein are provided in a sunscreen, sunblock, or suntan lotion composition.

In some cases, the source of radiation is a radiological dispersal devices, improvised nuclear devices, nuclear weapons, or nuclear power plant explosions. In some cases, the source of radiation is the sun, x-ray and other diagnostic machines, tanning beds, and other sources of ionizing radiation.

In some embodiments, the at least one alpha connexin polypeptide in a composition provided herein comprises the carboxy terminal-most 4 to 30 contiguous amino acids of an alpha Connexin. In some embodiments, the polypeptide consists of the carboxy terminal-most 5 to 19contiguous amino acids of the alpha Connexin. In some embodiments, the alpha Connexin is Connexin 37, Connexin 40, Connexin 43, or Connexin 45. In further embodiments, the polypeptide comprises the amino acid sequence selected from the group consisting of SEQ ID NO: 1, SEQ ID NO: 2, SEQ ID NO: 3, SEQ ID NO: 4, and SEQ ID NO: 5.

In some embodiments, the provided polypeptide can comprise an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the c-terminus of an alpha Connexin (ACT). Thus, in one aspect, the provided polypeptide comprises an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to SEQ ID NO:1, SEQ ID NO: 29, SEQ ID NO:30, SEQ ID NO:31, SEQ ID NO:32, SEQ ID NO:33, SEQ ID NO:34, SEQ ID NO: 35, SEQ ID NO:36, SEQ ID NO:37, SEQ ID NO:38, SEQ ID NO:39, SEQ ID NO:40,SEQ ID NO:41, SEQ ID NO: 89 or SEQ ID NO:90. As an example, provided is a polypeptide (SEQ ID NO:4) having 66% sequence identity to the same stretch of 9 amino acids occurring on the carboxy-terminus of human C×43 (SEQ ID NO:2).

In some embodiments, the polypeptide further comprises a cellular internalization sequence. In further embodiments, the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol). In some embodiments, the cellular internalization sequence is Antennapedia, and wherein the sequence comprises the amino acid sequence of SEQ ID NO:7. In some embodiments, the polypeptide comprises the amino acid sequence selected from the group consisting of SEQ ID NO:8, SEQ ID NO:9, SEQ ID NO:10, SEQ ID NO:11, and SEQ ID NO:12. In some embodiments, the alpha connexin polypeptide comprises a biotin at the N-terminus and/or the C-terminus of the polypeptide. In some embodiments, the composition comprises SEQ ID NO: 91.

Also provided are isolated nucleic acids encoding the polypeptides provided herein. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (e.g., a cDNA or a genomic or cDNA fragment produced by PCR, restriction endonuclease digestion, or chemical or in vitro synthesis). It also includes a recombinant DNA, which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, e.g., other types of RNA molecules or polypeptide molecules.

Thus, provided is an isolated nucleic acid encoding a polypeptide comprising the amino acid sequence SEQ ID NO:1, SEQ ID NO:2, SEQ ID NO:3, SEQ ID NO:4, SEQ ID NO:5, SEQ ID NO:7, SEQ ID NO:8, SEQ ID NO:9, SEQ ID NO:10, SEQ ID NO:11, or SEQ ID NO:12.

Thus, the provided nucleic acid can comprise the nucleic acid sequence SEQ ID NO:79,SEQ ID NO:80, SEQ ID NO:81, SEQ ID NO:82, SEQ ID NO:83, SEQ ID NO:84, SEQ ID NO:85,SEQ ID NO:86, SEQ ID NO:87 SEQ ID NO:88, or SEQ ID NO:89.

In some embodiments, the composition comprising the at least one alpha connexin polypeptide for use in a method for treating a radiation induced skin injury as provided herein is administered topically. In some embodiments, the composition further comprises hydroxyethylcellulose gel. In further embodiments, the hydroxyethylcellulose gel is present at a concentration of about 0.25% (w/w), about 0.5% (w/w), about 0.75% (w/w), about 1.00% (w/w), about 1.25% (w/w), about 1.5% (w/w) or about 2.0% (w/w).

In some embodiments, the compositions provided herein may be applied topically once per day, twice per day, three times per day, four times per day, five times per day, or more. In some embodiments, the compositions provided herein may be applied topically once every two days, once every three days, once every four days, once every four days, once every five days, once every six days, or once per week. In some embodiments, the compositions provided herein may be applied topically on an as-needed basis. For example, in some embodiments, the compositions provided herein may be applied during or after the manifestation or increasing severity of symptoms of the radiation induced injury as provided herein. In some embodiments, the compositions provided herein may be applied topically once per day for about 5, about 10, about 15, about 20, about 25, about 30, about 35, about 40, about 45, about 50, about 55, about 60, about 65, about 70, about 75, about 80, about 85, about 90, about 95, about 100, or more consecutive days. In some embodiments, the compositions provided herein comprise an ACT peptide provided herein and a gel and may be topically applied. In further embodiments, the compositions provided herein comprise SEQ ID NO: 91. In some embodiments, the compositions provided herein comprise SEQ ID NO: 91 and hydroxyethylcellulose gel, wherein the compositions are topically applied.

In some embodiments, the compositions provided herein include a topical gel termed “Granexin®”. In some embodiments, Granexin® comprises 1.25% hydroxyethyl cellulose gel and the ACT1 peptide. The chemical structure of the ACT1 peptide in Granexin® is: Biotin-Ahx-Arg-Gln-Pro-Lys-Ile-Trp-Phe-Pro-Asn-Arg-Arg-Lys-Pro-Trp-Lys-Lys-Arg-Pro-Arg-Pro-Asp-Asp-Leu-Glu-Ile-OH (SEQ ID NO: 91), wherein Ahx is L-2-aminohexanoic acid (6-aminohexanoic acid). In some embodiments, Granexin® further comprises one or more preservative, solvent, buffer agent, stabilizer, chelating agent, and/or any additional pharmaceutically acceptable excipient or carrier.

In certain embodiments the composition comprising the at least one alpha connexin polypeptide for use in a method for treating a radiation induced skin injury as provided herein is a topical formulation comprising the at least one alpha connexin polypeptide and hydroxyethylcellulose gel, wherein the hydroxyethylcellulose gel stabilizes the alpha connexin polypeptide. In certain embodiments, the hydroxyethylcellulose gel stabilizes the alpha connexin polypeptide so that after 3 months of storage at 5° C. at least 80%, 81%, 82%, 83%, 84%, 85%, 86%, 87%, 88%, 89%, 90%, 91%, 92%, 93%, 94%, 95%, 96%, 97%, 98%, 99%, or 100% of the alpha connexin polypeptide is detectable by analytical methods. In some embodiments, the alpha connexin polypeptide is present in the formulation at a concentration of about 0.0025% (w/w), of about 0.005% (w/w), of about 0.0075% (w/w), of about 0.010% (w/w), of about 0.015% (w/w), of about 0.020% (w/w), of about 0.025% (w/w), of about 0.030% (w/w), of about 0.035% (w/w), of about 0.040% (w/w), of about 0.045% (w/w), of about 0.050% (w/w), of about 0.055% (w/w), of about 0.060% (w/w), of about 0.065% (w/w), of about 0.070% (w/w), of about 0.075% (w/w), of about 0.080% (w/w), of about 0.085% (w/w), of about 0.090% (w/w), of about 0.095% (w/w), of about 0.100% (w/w), of about 0.150% (w/w), of about 0.200% (w/w), of about 0.250% (w/w), of about 0.500% (w/w), of about 0.750% (w/w), of about 1.00% (w/w), of about 1.50% (w/w), of about 2.00% (w/w), of about 2.50% (w/w), or of about 3.00% (w/w), or of about 3.50% (w/w), or of about 4.00% (w/w), or of about 4.50% (w/w), or of about 5.00% (w/w), or more. In one embodiment, the alpha connexin polypeptide is present in the formulation at a concentration of between about 0.005% (w/w) and about 1.00% (w/w).

In other embodiments, the drug product of the invention is a clear colorless gel which contains 0.0072% (w/w) (20 μM) of the ACT peptide, 0.018% (w/w) (50 μM) of the ACT peptide, 0.036% (w/w) (100 μM) of the ACT peptide, 0.072% (w/w) (200 μM) of the ACT peptide, or 0.36% (w/w) (1000 μM) of the ACT peptide. The ACT peptide may be dissolved in a semisolid dosage form that contains >0% water, >10% water, >20% water, >30% water, >40% water, >50% water, >60% water, >70% water, >80% water, or >90% water and 0.25% gelling agent (polymer), 0.55% gelling agent (polymer), 0.75% gelling agent (polymer), 1.00% gelling agent (polymer), 1.25% gelling agent (polymer), 1.50% gelling agent (polymer), 1.75% gelling agent (polymer), 2.00% gelling agent (polymer), 2.25% gelling agent (polymer), or 2.50% gelling agent (polymer), or 3.00% gelling agent (polymer), or 3.50% gelling agent (polymer), or 4.00% gelling agent (polymer), or 4.50% gelling agent (polymer), or 5 00% gelling agent (polymer).

The exact amount of the compositions required will vary from subject to subject, depending on the area of the radiation induced injury being treated. Thus, it is not possible to specify an exact amount for every composition. However, an appropriate amount can be determined by one of ordinary skill in the art using only routine experimentation given the teachings herein.

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual doctor in the event of any counterindications. Dosage can vary, and can be administered in one or more dose administrations daily, for one or several days. Guidance can be found in the literature for appropriate dosages for given classes of pharmaceutical products. The range of dosage largely depends on the application of the compositions herein, severity of condition, and its route of administration.

The peptides and/or other formulations embodying the invention including the use of one debridement or repeated debridements and can modulate cell migration and proliferation, thereby reducing inflammation, accelerating wound healing, reduce scarring and ultimately promote repair, regeneration and restoration of structure and function in all tissues. This acceleration is in comparison to a control. The control can be a standard of care known in the art for a particular radiation induced skin injury. The control can be a standard of care known in the art for a particular radiation induced skin injury that does not include use of an alpha connexin polypeptide and/or a debridement regimen as provided herein. The control can be a vehicle gel lacking the active pharmaceutical ingredient. Healing of wounds post-peptide application can involve significantly reduced fibrosis, consequently reduced scarring in skin wounds and fibrous patches in internal tissue injuries, promoting tissue regeneration, re-epithelialization, and restoring tissue and organ structure and function.

In some embodiments, the composition comprising the at least one alpha connexin polypeptide for use in a method for treating a radiation induced skin injury as provided herein can further comprise any known or newly discovered substance that can be administered to a wound, tissue injury, site of inflammation or cancer. For example, the provided composition can further comprise one or more of classes of antibiotics (e.g. Aminoglycosides, Cephalosporins, Chloramphenicol, Clindamycin, Erythromycins, Fluoroquinolones, Macrolides, Azolides, Metronidazole, Penicillin's, Tetracycline's, Trimethoprim-sulfamethoxazole, Vancomycin), steroids (e.g. Andranes (e.g. Testosterone), Cholestanes (e.g. Cholesterol), Cholic acids (e.g. Cholic acid), Corticosteroids (e.g. Dexamethasone), Estraenes (e.g. Estradiol), Pregnanes (e.g. Progesterone), narcotic and non-narcotic analgesics (e.g. Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), chemotherapy (e.g. anti-cancer drugs such as but not limited to Altretamine, Asparaginase, Bleomycin, Busulfan, Carboplatin, Carrustine, Chlorambucil, Cisplatin, Cladribine, Cyclophosphamide, Cytarabine, Dacarbazine, Diethylstilbesterol, Ethinyl estradiol, Etoposide, Floxuridine, Fludarabine, Fluorouracil, Flutamide, Goserelin, Hydroxyurea, Idarubicin, Ifosfamide, Leuprolide, Levamisole, Lomustine, Mechlorethamine, Medroxyprogesterone, Megestrol, Melphalan, Mercaptopurine, Methotrexate, Mitomycin, Mitotane, Mitoxantrone, Paclitaxel, pentastatin, Pipobroman, Plicamycin, Prednisone, Procarbazine, Streptozocin, Tamoxifen, Teniposide, Vinblastine, Vincristine), anti-inflammatory agents (e.g. Alclofenac; Alclometasone Dipropionate; Algestone Acetonide; alpha Amylase; Amcinafal; Amcinafide; Amfenac Sodium, Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate: Cormethasone Acetate; Cortodoxone: Decanoate; Deflazacort; Delatestryl; Depo-Testosterone; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Mesterolone; Methandrostenolone; Methenolone; Methenolone Acetate; Methylprednisolone Suleptanate; Momiflumate; Nabumetone; Nandrolone; Naproxen; Naproxen Sodium; Naproxol; Nimazone; Olsalazine Sodium; Orgotein; Orpanoxin; Oxandrolane; Oxaprozin; Oxyphenbutazone; Oxymetholone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Stanozolol; Sudoxicam; Sulindac; Suprofen; Talmetacin; Talniflumate; Talosalate; Tebufelone; Tenidap; Tenidap Sodium; Tenoxicam; Tesicam; Tesimide; Testosterone; Testosterone Blends; Tetrydamine; Tiopinac; Tixocortol Pivalate; Tolmetin; Tolmetin Sodium; Triclonide; Triflumidate; Zidometacin; Zomepirac Sodium), or anti-histaminic agents (e.g. Ethanolamines (like diphenhydramine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

In addition to the alpha connexin polypeptides and excipients, the composition provided herein may also include at least one additional therapeutic agent such as antimicrobial agents, anti-acne agents, anti-inflammatory agents, analgesic agents, anesthetic agents, antihistamine agents, antiseptic agents, immunosuppressants, antihemorrhagic agents, vasodilators, wound healing agents, anti-biofilm agents and mixtures thereof.

Examples of antimicrobial agents include penicillins and related drugs, carbapenems, cephalosporins and related drugs, erythromycin, aminoglycosides, bacitracin, gramicidin, mupirocin, chloramphenicol, thiamphenicol, fusidate sodium, lincomycin, clindamycin, macrolides, novobiocin, polymyxins, rifamycins, spectinomysin, tetracyclines, vanomycin, teicoplanin, streptogramins, anti-folate agents including sulfonamides, trimethoprim and its combinations and pyrimethamine, synthetic antibacterials including nitrofurans, methenamine mandelate and methenamine hippurate, nitroimidazoles, quinolones, fluoroquinolones, isoniazid, ethambutol, pyrazinamide, para-aminosalicylic acid (PAS), cycloserine, capreomycin, ethionamide, prothionamide, thiacetazone, viomycin, eveminomycin, glycopeptide, glyclyclycline, ketolides, oxazolidinone; imipenen, amikacin, netilmicin, fosfomycin, gentamycin, ceftriaxone, Ziracin, Linezolid, Synercid, Aztreonam, and Metronidazole, Epiroprim, Sanfetrinem sodium, Biapenem, Dynemicin, Cefluprenam, Cefoselis, Sanfetrinem celexetil, Cefpirome, Mersacidin, Rifalazil, Kosan, Lenapenem, Veneprim, Sulopenem, ritipenam acoxyl, Cyclothialidine, micacocidin A, carumonam, Cefozopran and Cefetamet pivoxil.

Examples of topical anti-acne agents include adapalene, azelaic acid, benzoyl peroxide, clindamycin and clindamycin phosphate, doxycycline, erythromycin, keratolytics such as salicylic acid and retinoic acid (“Retin-A”), norgestimate, organic peroxides, retinoids such as isotretinoin and tretinoin, sulfacetamide sodium, and tazarotene. Particular anti-acne agents include adapalene, azelaic acid, benzoyl peroxide, clindamycin (e.g., clindamycin phosphate), doxycycline (e.g., doxycycline monohydrate), erythromycin, isotretinoin, norgestimate, sulfacetamide sodium, tazarotene, etretinate and acetretin.

Examples of antihistamine agents include diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride, and the like. Examples of local anesthetic agents include dibucaine hydrochloride, dibucaine, lidocaine hydrochloride, lidocaine, benzocaine, p-buthylaminobenzoic acid 2-(die-ethylamino) ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine and dyclonine hydrochloride.

Examples of antiseptic agents include alcohols, quaternary ammonium compounds, boric acid, chlorhexidine and chlorhexidine derivatives, iodine, phenols, terpenes, bactericides, disinfectants including thimerosal, phenol, thymol, benzalkonium chloride, benzethonium chloride, chlorhexidine, povidone iode, cetylpyridinium chloride, eugenol and trimethylammonium bromide.

Examples of anti-inflammatory agents include nonsteroidal antiinflammatory agents (NSAIDs); propionic acid derivatives such as ibuprofen and naproxen; acetic acid derivatives such as indomethacin; enolic acid derivatives such as meloxicam, acetaminophen; methyl salicylate; monoglycol salicylate; aspirin; mefenamic acid, flufenamic acid, indomethacin, diclofenac; alclofenac; diclofenac sodium; ibuprofen; ketoprofen; naproxen; pranoprofen; fenoprofen; sulindac; fenclofenac; clidanac; flurbiprofen, fentiazac; bufexamac; piroxicam; phenylbutazone; oxyphenbutazone; clofezone; pentazocine; mepirizole; tiaramide hydrochloride; steroids such as clobetasol propionate, bethamethasone dipropionate, halbetasol proprionate, diflorasone diacetate, fluocinonide, halcinonide, amcinonide, desoximetasone, triamcinolone acetonide, mometasone furoate, fluticasone proprionate, betamethasone diproprionate, triamcinolone acetonide, fluticasone propionate, desonide, fluocinolone acetonide, hydrocortisone vlaerate, prednicarbate, triamcinolone acetonide, fluocinolone acetonide, hydrocortisone and others known in the art, predonisolone, dexamethasone, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, fluocinonide, topical corticosteroids, and may be one of the lower potency corticosteroids such as hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, or methylprednisolone, or may be a higher potency corticosteroid such as clobetasol propionate, betamethasone benzoate, betamethasone dipropionate, diflorasone diacetate, fluocinonide, mometasone furoate, triamcinolone acetonide.

Examples of analgesic agents include alfentanil, benzocaine, buprenorphine, butorphanol, butamben, capsaicin, clonidine, codeine, dibucaine, enkephalin, fentanyl, hydrocodone, hydromorphone, indomethacin, lidocaine, levorphanol, meperidine, methadone, morphine, nicomorphine, opium, oxybuprocaine, oxycodone, oxymorphone, pentazocine, pramoxine, proparacaine, propoxyphene, proxymetacaine, sufentanil, tetracaine and tramadol.

Examples of anesthetic agents include alcohols such as phenol; benzyl benzoate; calamine; chloroxylenol; dyclonine; ketamine; menthol; pramoxine; resorcinol; troclosan; procaine drugs such as benzocaine, bupivacaine, chloroprocaine; cinchocaine; cocaine; dexivacaine; diamocaine; dibucaine; etidocaine; hexylcaine; levobupivacaine; lidocaine; mepivacaine; oxethazaine; prilocalne; procaine; proparacaine; propoxycaine; pyrrocaine; risocaine, rodocaine; ropivacaine; tetracaine; and derivatives, such as pharmaceutically acceptable salts and esters including bupivacaine HCl, chloroprocaine HCl, diamocaine cyclamate, dibucaine HCl, dyclonine HCl, etidocaine HCl, levobupivacaine HCl, lidocaine HCl, mepivacaine HCl, pramoxine HCl, prilocalne HCl, procaine HCl, proparacaine HCl, propoxycaine HCl, ropivacaine HCl, and tetracaine HCl.

Examples of antihemorrhagic agents include thrombin, phytonadione, protamine sulfate, aminocaproic acid, tranexamic acid, carbazochrome, carbaxochrome sodium sulfanate, rutin and hesperidin.

Also provided herein is an animal model for radiation induced skin injuries such as, for example, cutaneous radiation injury (CRI). In some cases, the animal model provided herein is in compliance with the United States Food and Drug Administration's (FDA) animal rule that serves as a regulatory pathway for medical countermeasures (MCMs) against Chemical, Biological, Radiological, and Nuclear (CBRN) threats where efficacy trials in humans are not feasible. The animal model provided herein can be implemented in a porcine animal. In some cases, due to similarities of pig and human skin, the pig is considered to be the optimal large animal model to study radiation induced skin injuries such as, for example, CRI. As provided herein, the porcine radiation induced skin injury model (e.g., Yorkshire swine) utilizes pig skin that is irradiated with an electron beam using a clinical linear accelerator. The electron beam can be shaped using a custom made Cerrobend shield with a custom lead collimator. The lead collimator can be configured to possess 4 holes of d=4 cm, spaced 4 cm apart. Eight millimeters of tissue bolus material can be placed on the animal's side, and the irradiation sites positioned to obtain a Source to Surface (SSD) of 100 cm. Electrons can be delivered at measured output dose of 35-60 Gy. The animal can be rotated, and the irradiation can be repeated, for a total of 8 irradiated skin sites on each animal.

Suitability of the aforementioned animal model as a model for radiation induced skin injury can entail: (a) when signs and symptoms of a radiation induced injury manifest, animals can be treated daily with systemic antibiotics (e.g., amoxicillin) and tramadol for pain management; (b) cleaning (e.g., daily) of the injury site with a topical cleanser and gauze; (c) at onset of erythema, desquamation, necrosis or any combination thereof within irradiated wounds, surgical debridement can be initiated. In some cases, animals can be assessed routinely (e.g., every 4-6 days) for the clinical need for debridement. For example, in some cases, the animals can be sedated every 5 days and each irradiated skin site assessed for clinical need for debridement. If necrotic tissue is present, it can be removed. In some cases, the model further entails euthanizing the animals after a defined period post irradiation (e.g., at 120 days post irradiation) and performing clinical evaluations. Said clinical evaluations can comprise measurements of wound size, and histopathology in order to confirm that the irradiation parameters recapitulated a beta particle radiation exposure that results in a CRI with a natural history that mirrors that seen in humans.

Use of the aforementioned animal model for treating radiation induced skin injury following irradiation as provided herein can entail: (a) when signs and symptoms of a radiation induced injury manifest, animals can be treated daily with systemic antibiotics and tramadol for pain management; (b) cleaning (e.g., daily) of the injury site with a topical cleanser and gauze; (c) at onset of erythema, desquamation, necrosis or any combination thereof within irradiated wounds, surgical debridement can be initiated; and (d) administration of a composition provided herein comprising at least one alpha connexin polypeptide as provided herein on a defined schedule. In some cases, animals can be assessed routinely (e.g., every 4-6 days) for the clinical need for debridement. For example, in some cases, the animals can be sedated every 5 days and each irradiated skin site can be assessed for clinical need for debridement. If necrotic tissue is present, it can be removed. In some cases, the defined schedule for step (d) is daily. In some cases, steps (c) and (d) are repeated for a defined period of time, which can be at least 120 days post irradiation. In some cases, the model further entails euthanizing the animals after a defined period post irradiation (e.g., at 120 days post irradiation) and clinical evaluations performed. Said clinical evaluations can comprise measurements of wound size, and histopathology in order to evaluate whether or not treatment with the composition comprising the at least alpha connexin polypeptide as provided herein lead to treatment of the wound as compared to standard of care treatment. The standard of care treatment can be any treatment of a radiation induced skin injury known in the art that does not entail use of an alpha connexin polypeptide as provided herein.

### EXAMPLES

The present disclosure is further illustrated by reference to the following Examples. However, it should be noted that these Examples, like the embodiments described above, are illustrative and are not to be construed as restricting the scope of the invention in any way.

### Example 1—Development of a Reproducible and Military Relevant Yorkshire Swine Model of Radiation Burns for Evaluation of Medical Countermeasures (MCMs)

### Background

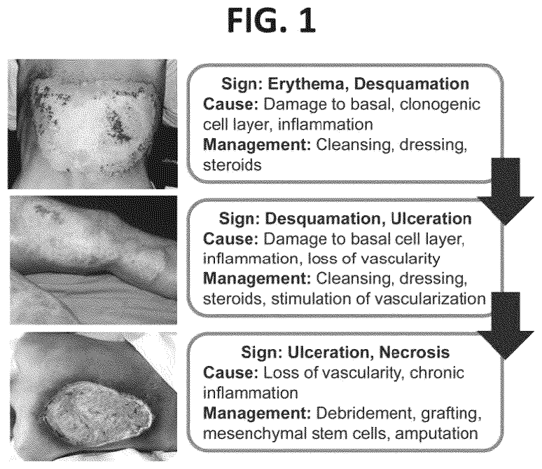

A nuclear or radiological incident could lead to exposure of civilians to high doses of radioactive material. This exposure could be due to any number of risks such as, for example, Improvised Nuclear Devices (IND) or nuclear detonation, Radiological Dispersal Devices (RDDs) or ‘Dirty Bombs’, nuclear power plant incidents in combat zones, and improperly disposed radiological equipment encountered in combat. Cutaneous radiation injuries (CRI), also known as radiation burns, can be caused by external exposure to high doses of ionizing radiation. As shown in FIG. 1, radiation induced injuries such as CRI manifest with dose dependent severities ranging from erythema and desquamation to severe, full-thickness wounds.

Medical management of radiation induced injuries is resource intensive. Moreover, there are no FDA approved drugs that improve outcomes in radiation induced skin injuries.

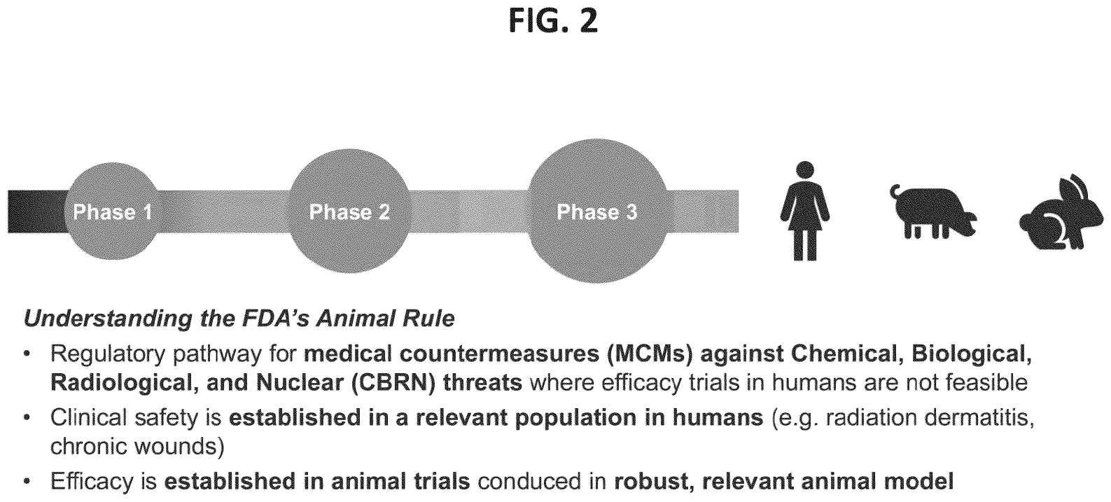

In view of the foregoing, a well characterized and clinically relevant animal model of CRI is necessary for product approval, especially under the FDA's Animal Rule (see FIG. 2).

### Objective

Accordingly, the goal of this Example was to develop a reproducible animal model of CRI in the Yorkshire swine. Additionally, it was the goal of this Example to develop supportive care measures that aligned with mass casualty relevant treatment scenarios, which were deemed essential to include in animal models intended to assess efficacy of MCMs for CRI.

### Materials and Methods

Due to similarities of porcine and human skin, the pig is considered to be the optimal large animal model to study cutaneous radiation injury. Studies have shown that dermal injury pathologies in pigs show the same natural history as is seen in humans. As such, pigs are an ideal species to assess the therapeutic potential of topical agents in cutaneous disease. The total number of animals is the number needed to properly characterize responses related to skin-focused irradiation and Test Article administration and thus to meet experimental objectives.

### Inclusion and Exclusion Criteria

Animals were assessed for eligibility for study enrollment on the day of irradiation.

### Inclusion Criteria

Yorkshire swine (Sus Scrofa domestica) that meet the following criteria were included in the study:
1. Young adult male and female Yorkshire swine: weight between 20-30 kg on the day of irradiation; age between 2-4 months;
2. In good health and free of malformations and clinical signs of disease;
3. No skin injury on the paraspinal dorsal skin surface between the scapula and hip bones;
4. Negative coccidia test results

### Exclusion Criteria

Animals were excluded from the study if there was any evidence of poor health, illness, or injury, which in the judgment of the Study Director made the animal a poor candidate for the study.

Given the importance of beta-type irradiation induced CRI in nuclear incidents, irradiation parameters were established to mimic characteristic depth dose drop off patterns associated with beta-type irradiation exposures. Water phantom exposures and in vivo biodosimetry confirmed radiation dosimetry. Yorkshire swine were anesthetized and exposed to a single fraction of ionizing radiation using a Linear Accelerator to 8 separate sites on the paraspinal dorsal skin surface. Irradiated skin sites were evaluated daily for presence of dermal injury. Planimetric measurements of wound size were performed from clinical photographs and histopathology conducted at terminal necropsy.

2 males and 4 females were used for acclimation and irradiation. Animals were acclimated for a minimum of ten days prior to study enrollment. During the acclimation period, animals were acclimated to routine handling by study personnel.

Animals were socially housed during acclimation. Animals were individually housed following irradiation. Primary enclosure complies with the Animal Welfare Act and recommendations outlined in the Guide for the Care and Use of Laboratory Animals (8th edition). Bedding were used. Pens were cleaned daily and sanitized every other week. Feeders were sanitized every other week.

Housing conditions were maintained except for times when animals were separated for study-specific procedures.

PMI LabDiet® 5K99 Certified Lab Mini Pig Diet was provided twice per day adjusted for body weight per veterinary recommendations. Fresh water was provided ad libitum via automated watering.

Animals were fasted as required for study-specific procedures. No contaminants were present at levels that would interfere with the outcome of the study. Food and water analysis records were maintained in the Testing Facility records.

### Environmental Conditions

The animals were housed in a temperature and humidity-controlled environment. The targeted range for animal holding room temperature is 64-81° F. (18-27° C.) with 30 to 70% relative humidity. A 12-hour light/dark cycle was maintained except when interrupted by study activities and was documented.

Fruits, vegetables, treats, as well as enrichment devices were provided throughout the course of the study.

### Identification

Animals were identified by a unique number on an ear tag provided at vendor.

### Blinding

Technical Research staff, Veterinary staff, Pathology staff, the Study Director, staff conducting planimetry and any other staff collecting animal observation data, or involved in the medical care management of the animals were blinded to treatment group until the database was locked.

Test and Control Articles were labeled for blind administration. Test and Control Articles were identical in appearance and differed only by inclusion of the active ingredient in the test article.

### Randomization and Blinded Group Assignment

To meet study objectives and allow monitoring of adequacy of the protocol defined blinding procedure in maintaining the blind throughout the study, there was an unblinded study team.

The unblinded study team consisted of:
Unblinded drug manager (CA/TA blinding and dispensation)
Unblinded QC representative
Unblinded statistician
Sponsor Study Monitor

As defined above, all unblinded personnel were independent of medical care management of the animals. The study statistician were unblinded for the duration of the study. Statistical analyses were conducted by the unblinded statistician. The unblinded team were not involved with any study procedures (including dosing of control article and test article). To maintain the blind, the unblinded statistician generated 6×3 digit Animal IDs. The unblinded statistician were randomly assigned the animal IDs to either test article or control article. These unblinded assignments were transferred to the unblinded drug manager, who then re-labeled the required number of test article and control article tubes with the appropriate blinded animal IDs. A blinded animal ID were assigned to each enrolled animal in a randomized order. Tubes labelled with each blinded animal ID were made available to the blinded team members for application, as per the study protocol. The unblinded assignments (control or test article) were shared with the study team post database lock

### Test Article

Active Pharmaceutical Ingredient: alpha Connexin carboxy-terminal 1 (aCT1) peptide (SEQ ID NO: 9). In this study, the aCT1 peptide was present in a composition that was a topical gel termed Granexin Gel (0.036%, 15 g). The chemical structure of the ACT1 peptide in Granexin® was: Biotin-Ahx-Arg-Gln-Pro-Lys-Ile-Trp-Phe-Pro-Asn-Arg-Arg-Lys-Pro-Trp-Lys-Lys-Arg-Pro-Arg-Pro-Asp-Asp-Leu-Glu-Ile-OH (SEQ ID NO: 91), wherein Ahx is L-2-aminohexanoic acid (6-aminohexanoic acid). In some cases, Granexin® further comprised one or more preservative, solvent, buffer agent, stabilizer, chelating agent, and/or any additional pharmaceutically acceptable excipient or carrier.

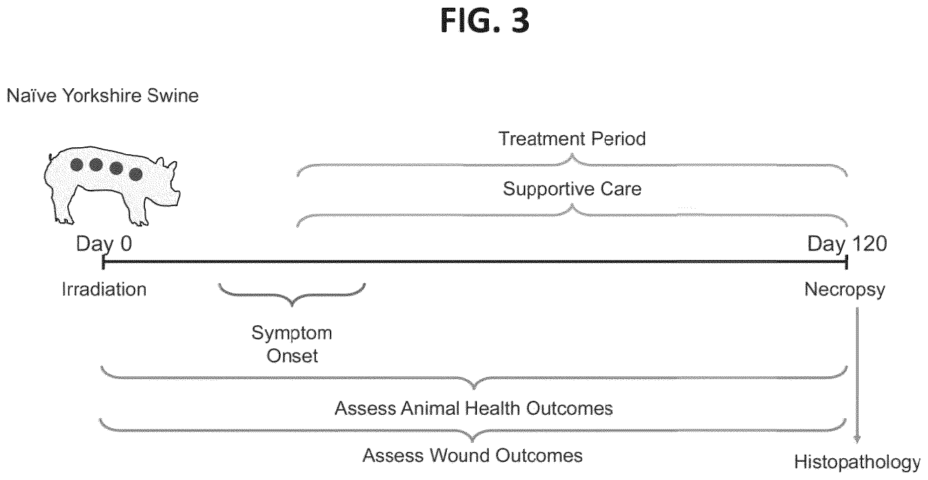

### Dosing and Route of Administration (see FIG. 3)

Test and Control Articles were maintained on refrigerated gel packs, opened just before application, and topically applied to irradiated skin sites in an even, uniform layer to completely cover the immediate irradiation site and/or wound bed and just outside the immediate irradiation site and/or wound bed without overlap between sites.

### Duration of Therapy

Topical dose administration were initiated at the onset of persistent erythema (defined as redness that occurs within the irradiation site, after the initial 7 days post-irradiation and persists ≥24 hours) or presence of desquamation or ulceration within the irradiation site. Test (aCTI in Granexin®) and Control Articles (Granexin® alone) were administered once daily. Test or Control Article administration was topped once the wound had closed or day of terminal necropsy, whichever occurred first.

### Animal Irradiation

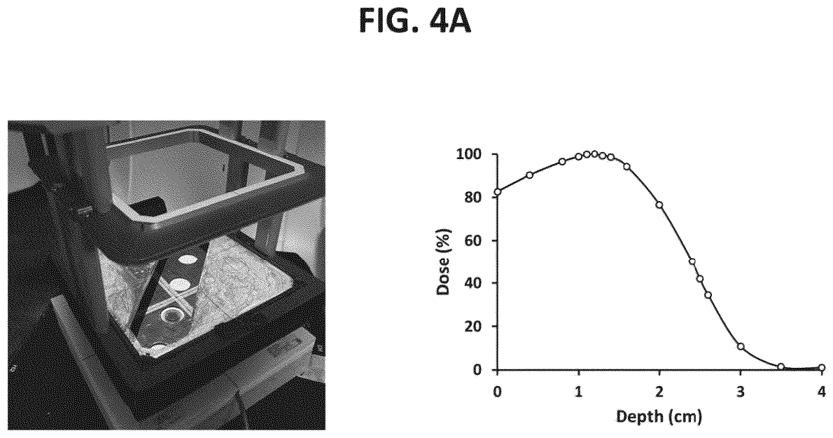

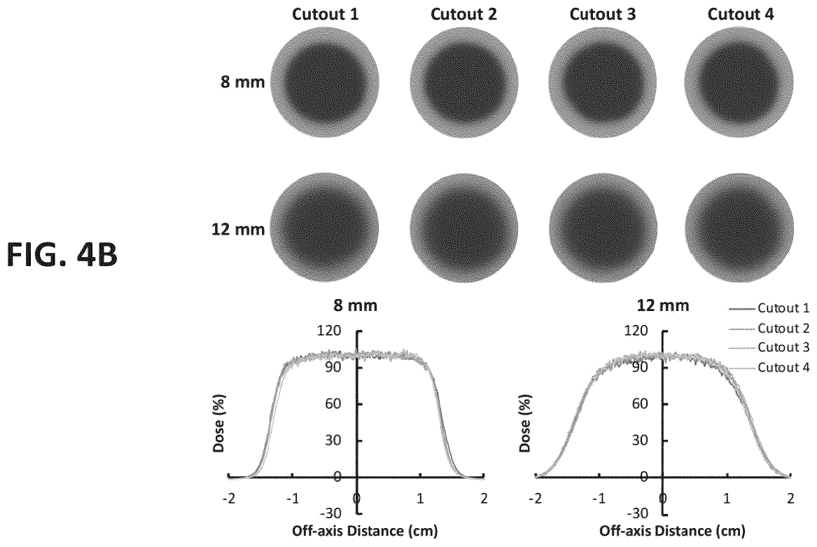

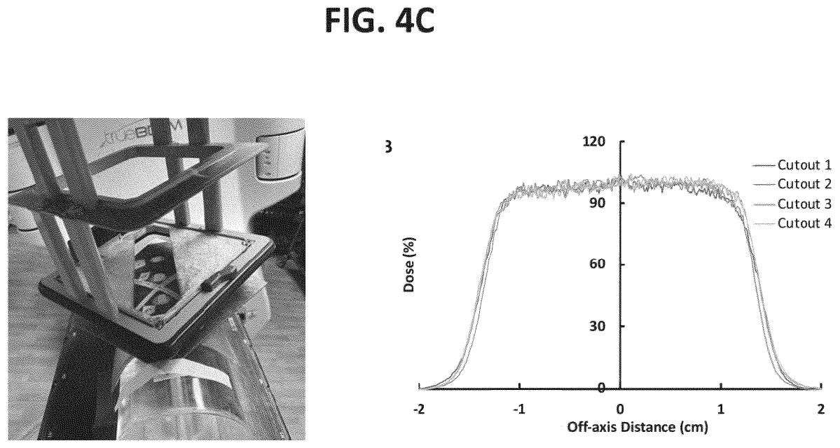

15.1 Radiation Geometry (see FIG. 4A-4C)

Each animal was irradiated at a total of 8 circular skin sites with a diameter of 4 cm. Four sites on each hemi-dorsal surface in the area between the scapula and hip bones was irradiated.

### Irradiation Preparation

Animals were fasted as necessary prior to sedation to reduce the risk of aspiration of emesis while anesthetized.

### Skin Preparation

Hair was removed by shaving from the dorsal surface in the area between the scapula and hip bones prior to irradiation. Care was taken to avoid any nicks or cuts.

On the day of irradiation, the area between the scapula and hip bones was washed with 0.0120% hypochlorous acid (e.g., Vetericyn) and gently patted dry.

### Anesthesia

Prior to transport to the LINAC, animals will be anesthetized with Telazol, BAM [Butorphanol (2.5 mg/mL)/Azaperone (0.8 mg/mL)/Medetomidine (1.0 mg/mL)], or Telazol/Xylazine. Using Isoflurane, animals were kept anesthetized throughout the irradiation procedure. A reversal agent was used as necessary at the conclusion of the irradiation procedure. Eyes were lubricated with an ophthalmic lubricant as needed throughout the irradiation procedure.

### Irradiation Site Identification

After irradiation, 4-6 marks per site (e.g., dots) were applied using permanent marker to delineate the irradiation area. Permanent marker marks to delineate the irradiated area were reapplied, as necessary. The letter designation of each site (A-H) were applied by tattoo using a tattoo gun. All tattoos were outside the irradiation site.

### Irradiation Procedure

Each animal received irradiation exposure of 45 Gy delivered to each skin site using a Varian TrueBeam® linear accelerator (LINAC) with 6 MeV electron source. Four sites on each hemidorsal surface in the area between the scapula and hip bones was irradiated for a total of 8 skin sites on each animal. A custom Cerrobend cutout was used to collimate the electron beam. Lead shielding with four circular 4 cm cutouts was used for skin collimation. Tissue equivalent bolus material (8 mm) was placed on the skin and under the lead shielding to ensure dose uniformity. The irradiation dose of 45 Gy at dmax was expected to result in dermal injury of all sites within the 90-day duration of the study. The number of monitor units needed to deliver the prescribed dose was calculated based on the output factors of the 4 cm circular field size and scaled based on the daily output of the LINAC.

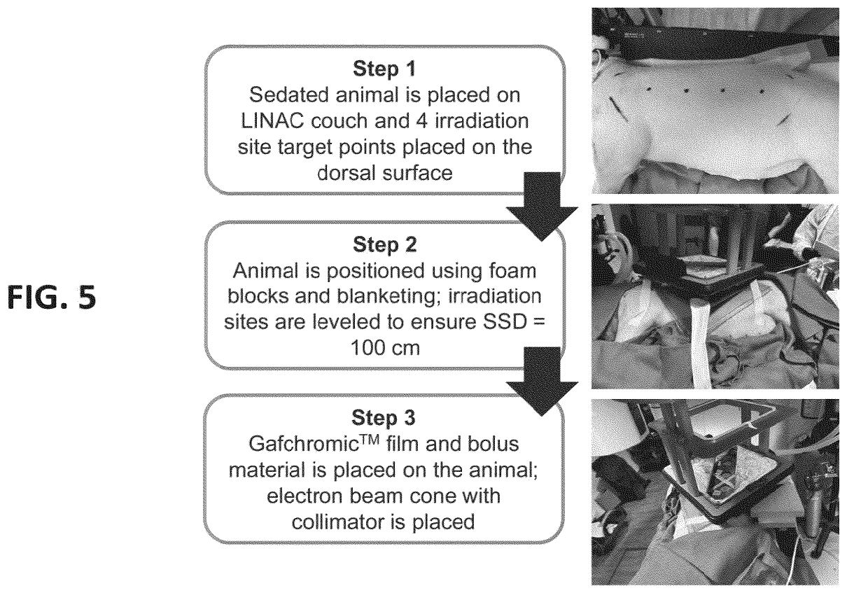

Sedated and immobilized animals was placed on the LINAC treatment couch (see FIG. 5, step 1). Props, such as foam blocks, were used as necessary to position animals to ensure the dorsal area to be irradiated was level (see FIG. 5, step 2). For in vivo dosimetry, Gafchromic™ film were taped to at least one irradiation site (see FIG. 5, step 3). The LINAC gantry was adjusted as necessary to ensure the electron beam was perpendicular to the irradiated sites, and a collimator angle selected to align the collimated beam in parallel with the four circular irradiation sites. The couch position was adjusted as necessary to ensure the appropriate SSD when it is centered in the radiation field.

### In-Run Biodosimetry

The LINAC was calibrated annually following the AAPM Task Group 51 protocol for clinical reference dosimetry of high energy photons and electrons, and the calibration record is maintained at UMMC Department of Radiation Oncology (located at 22 S. Greene St, Baltimore, MD 21201). Monthly and daily QA was performed following the recommendations of AAPM Task Group 142, and reports were retained at the Test Facility. The output of the LINAC was verified daily using a Sun Nuclear DailyQA3 device, and a copy of the results were included in the study data for each irradiation day. After irradiation, the Gafchromic™ film was collected, placed in opaque envelopes, and read at the test facility to measure the dose delivered to each irradiation site.

### Clinical Observations

Clinical observations, excluding irradiation sites were performed every 7 days ±2 days, beginning on the first week of animal arrival to the facility. Clinical observations included: body temperature, respiration rate, SpO2, and heart rate. Additional clinical parameters or observations were performed to properly monitor the animal's health condition, if recommended by a veterinarian.

### Skin Site Evaluations

Beginning on Day 0 and continuing until terminal necropsy, observation and documentation of the presence of dermal injury (erythema, desquamation, ulceration, necrosis) was conducted daily on each irradiation site by trained staff (see FIG. 3). For this study, erythema was defined as any redness of the irradiation site; desquamation was defined as any skin slough within the irradiation site; ulceration was defined as any open wound within the irradiation site; necrosis was defined as presence of any non-viable tissue within the irradiation site.

### Wound Closure Evaluations

Wounds within the irradiation site were assessed daily for complete closure (presence of 100% epithelialization) beginning on Day 0 and continuing until terminal necropsy (see FIG. 3). Sites were documented as either “open” or “closed”. An open wound was defined as any macroscopically visible loss in skin integrity.

### Body Weight

Animals were weighed prior to randomization and on Day 0, then a minimum of once every 7±2 days thereafter throughout the study. Body weight was taken on the day of scheduled necropsy. Additional body weights were taken, if necessary (e.g. if body weight is ≥25 % from baseline, the animal was weighed the next two consecutive days for evaluation of euthanasia criteria)

### Photography

At least 2 photographs per irradiation site were taken using a 3D LifeViz Micro Camera per the Image Procedure document, provided by Quantificare. The intent was to obtain at least 2 high quality images of each irradiation site. The settings on the camera (as listed in the Image Procedure, provided by Quantificare) were pre-set by Quantificare and were not changed. Camera calibration was confirmed by Quantificare prior to study initiation.

If hair covered the site, the hair was clipped as necessary; care was taken to avoid disturbing the injury.

On Day 0, photographs were taken pre-and post-irradiation. Subsequently, photographs were taken every 5 days, prior to application of Test Article or Control Article, as applicable. Once debridement was initiated for an individual irradiation site photographs of the irradiation site were taken on days of scheduled debridement (every 5 days ±1) pre-and post-debridement. Irradiation site photographs were taken on the day of documented wound closure, and prior to terminal necropsy. Prior to taking photographs, each irradiation site was cleaned, and exudate or pooled blood was removed by gently wiping the area with gauze sprayed with 0.0120% hypochlorous acid (e.g. Vetericyn®) and/or patting the site dry with gauze, as needed. Photographs were taken before and after debridement procedures, while animals were sedated. If necessary. gauze and light pressure were applied to stop excess bleeding. Additional photographs were taken as needed in order to obtain at least 2 good photographs. The 2 best photographs were uploaded to the Dermapix platform, per the Image Procedure. If an event occurs that the Study Director deemed clinically significant, additional photos of the irradiation site were taken outside of scheduled photography days.

Copies of 2 photographs per animal, in electronic format, were uploaded to Quantificare's secure image database (Dermapix). Pictures were used by the Principal Investigator for Planimetry for evaluation.

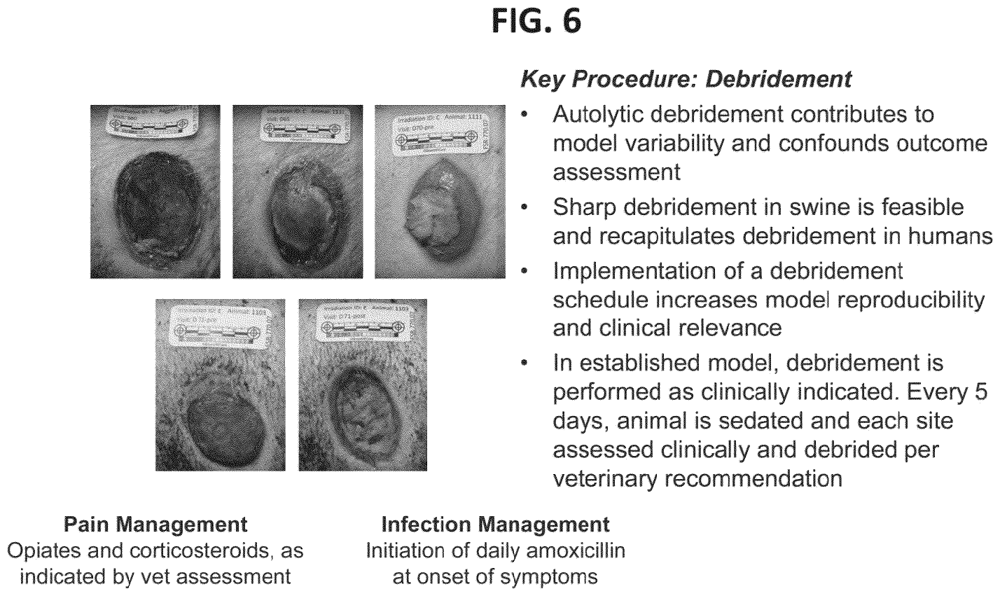

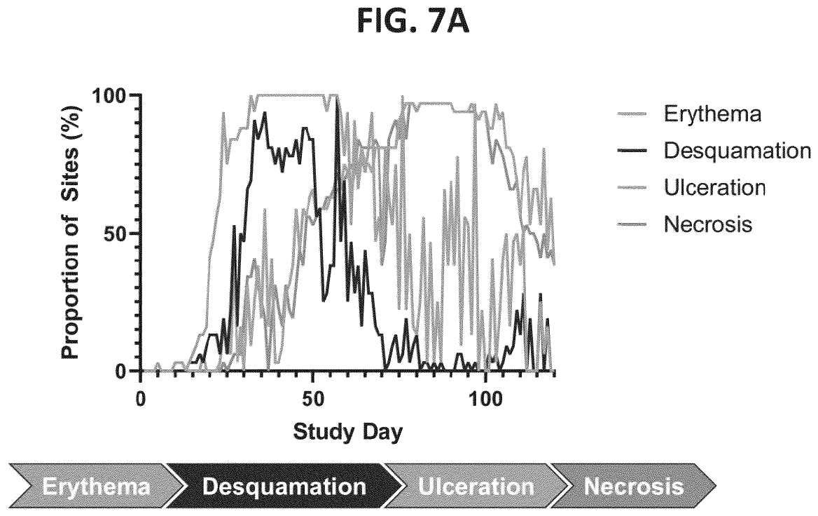

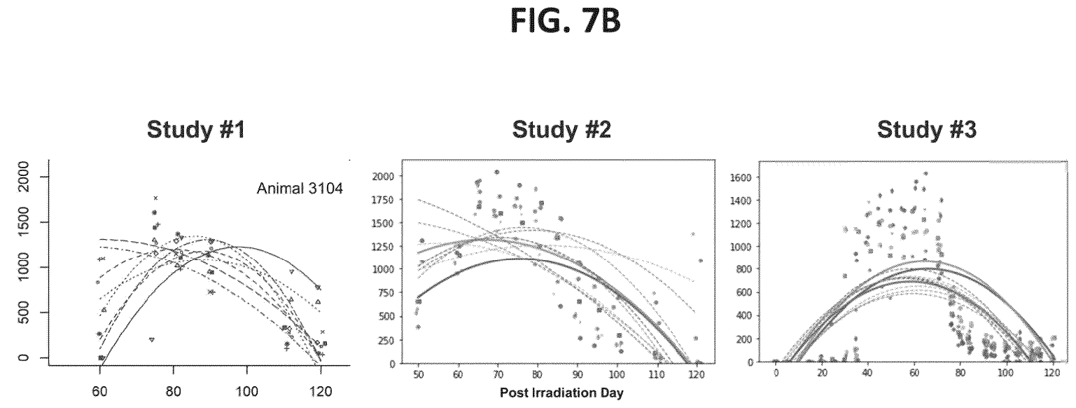

### Supportive Care (see FIG. 6)

### Management of Pain and Discomfort

Any animal showing signs of excessive scratching due to irritation and/or itching were brought to the attention of the Study Director and/or veterinary staff. If deemed necessary, diphenhydramine and/or buprenorphine was provided.

Analgesics (Tramadol, PO, 2-4 mg/kg BID) were administered to any animal observed to have persistent erythema (defined as redness that occurs after the first 7 days post-irradiation and persists ≥24 hours). Animals were prescribed additional analgesics per veterinary recommendation.

### Management of Infection

Animals were administered antibiotics (Amoxicillin, PO, 10 mg/kg BID) beginning on post irradiation Day 35. Antibiotic administration continued until the end of in-life (Day 90).

Any animal with an irradiation site that developed an odor or infection was brought to the attention of the veterinary staff. If an irradiation site developed an odor or infection prior to initiation of oral antibiotics as described above, the animal was administered antibiotics (Amoxicillin, PO, 10 mg/kg BID).

### Irradiation Site Bandaging

Beginning at first application of Test or Control articles, individual skin irradiation sites were covered with Tegaderm™ or comparable occlusive film dressing. Dressings were changed daily, after application of Test or Control articles. Dressings were reapplied as necessary in order to keep irradiation sites covered. A tubular net or comparable secondary dressing was applied over the dorsal area, to cover all irradiation sites on the animal.

### Irradiation Site Cleaning

Each day following dressing removal and prior to treatment administration, irradiation sites were cleaned by gently wiping the area with gauze sprayed with 0.0120% hypochlorous acid. Any remaining Test or Control article and/or exudate was gently wiped away and sites were patted dry prior to subsequent application of Test or Control articles. Cleaning was repeated as necessary to ensure the wound was clear of exudates and/or pooled blood prior to photography and application of test or control articles.

### Debridement

Surgical debridement was conducted on each irradiation site by research staff or under direct veterinary supervision, at onset of tissue necrosis (±2 days) and continuing at 5 day (±1 day) intervals. In this protocol, onset of tissue necrosis was defined as two consecutive observations of necrosis within the irradiation site during skin site evaluations. For debridement, animals were sedated (e.g., Telazol, BAM, or isoflurane) and each irradiation site was surgically or nonsurgically debrided, as clinically indicated. Irradiation sites for which debridement was not clinically indicated was cleaned with 0.0120% hypochlorous acid-soaked gauze. Topical or injectable analgesic (e.g., Lidocaine) was applied to the irradiation site for debridement as necessary. Hair surrounding the irradiation site was removed while animals are sedated for debridement.

Debridement notes were recorded at every debridement. Veterinary notes/comments from the supervising debridement procedures were documented and included but were not limited to: eschar thickness, wound depth post-debridement, documentation of partial or full thickness injury, and description of tissue remaining post-debridement (i.e., fibrotic, vascular, necrotic tissue). The presence of necrotic tissue post-debridement was noted, if applicable.

### Histopathologic Examination

Collected skin issues were trimmed, processed to paraffin blocks, sectioned, and stained with hematoxylin and eosin (H&E). Histopathological examination was performed by a board-certified veterinary pathologist. Images were captured as necessary for illustration or consultation purposes following approval from the Study Director. At a minimum, skin specimens were evaluated for changes in epidermis, dermis and subcutis as indicated in Table 1 and graded as Grade 0 (not present), Grade 1 (minimal, lesion involves less than 1% of structure), Grade 2 (mild, lesion involves at least 1% but less than 10% of structure), Grade 3 (moderate, lesion involves 10 to 50% of structure), Grade 4 (marked, lesion involves more than 50% of structure).

TABLE 1

| Epidermis | Dermis | Subcutis |
|---|---|---|
| Ulceration | Necrosis | Fibrin |
| Acanthosis | Infiltration, mixed | Fibroplasia |
| | inflammatory cells | |
| Parakeratosis | Fibroplasia | Infiltration, mixed |
| | | inflammatory cells |
| Infiltration, mixed | — | Necrosis |
| inflammatory cells | | |

### Study Assessments

Number of irradiation sites impacted by protocol deviations
Number of animals impacted by protocol deviations
Evaluation of accuracy of delivered dose from prescribed irradiation dose
Evaluation of adequacy of debridement of irradiation site wounds, as verified by a central evaluator from irradiation site photographs
Evaluation of adequacy of phenotypic evaluations of irradiation site wounds, as verified by a central evaluator from irradiation site photographs
Irradiated skin sites progress in accordance with natural history of CRI
Evaluation of deviations in Test Article treatment initiation and administration
Exploratory assessment of efficacy of Test Article in CRI wound treatment

### Adequacy of Irradiation Exposure and Biodosimetry

Commissioning of the LINAC was performed prior to study initiation and commissioning records retained with the Test Facility. For each day of irradiation exposures, LINAC output factor was documented. Upon study completion, output factor corrections were reviewed and compared against commissioning data to ensure acceptable performance characteristics for the LINAC were met. During each irradiation exposure, Gafchromic™ film was placed in at least one irradiated skin site for in vivo biodosimetry. Gafchromic™ film outputs for each irradiation exposure were reviewed and compared against the prescribed irradiation dose of 45 Gy.

TABLE 2
Schedule of Events

| | Time/Frequency | | | | | |
|---|---|---|---|---|---|---|
| | | | | Days | | Post- |
| Assay or Procedure | Pre-IR | D0 | Days 0-69 | 70-89 | Day 90 | Mortem |

| | | | | | |
|---|---|---|---|---|---|
| Inclusion/Exclusion Criteria | X | X | — | — | — |
| Irradiation of Skin Sites | — | X | — | — | — |
| Cageside Observations | 3x per week | Twice | Twice Daily¹ | Once | — |
| Clinical Observations | Every 7 ± 2 days | X | Every 7 +/− 2 days¹ | X | — |
| Body Weight | Every 7 ± 2 days | X | Every 7 ± 2 days | X | — |
| Skin Site Evaluation | — | X | Daily | X | — |
| Wound Closure Evaluation | — | X | Daily | X | — |
| Test and Control Article | — | — | Once daily per protocol | — | — |
| Administration | | | Section 14. | | |
| Photographs | — | Pre- and | Every 5 days until initiation of | Before | |
| | | Post- | debridement. Upon initiation | necropsy | |
| | | Irradiation | of debridement, Pre- and Post- | | |
| | | | debridement and on day of | | |
| | | | documented wound closure. | | |
| Debridement | — | — | At onset of tissue necrosis | — | — |
| | | | (+/−2 days) and subsequently | | |
| | | | every 5 +/− 1 days | | |
| Gross Necropsy and Tissue | — | — | — | X | — |
| Harvest | | | | | |
| Histology | — | — | — | — | X |

¹More frequent observations may be performed for animals meeting criteria.
— = Not performed
X = Performed

### Results and Conclusions

Overall, this study showed that a reproducible animal model of cutaneous radiation injuries for evaluation of medical countermeasures to enable regulatory approval under the FDA's Animal Rule has been developed. Within this model, radiation physics that mimic dose depth penetration of beta-type radiation exposure (primary cause of CRI) have been created and validated. Moreover, supportive care practices relevant to military care and mass casualty settings have been established. Further, the feasibility of conducting model under Good Laboratory Practice (GLP) regulations has been established in three GLP studies conducted to date.

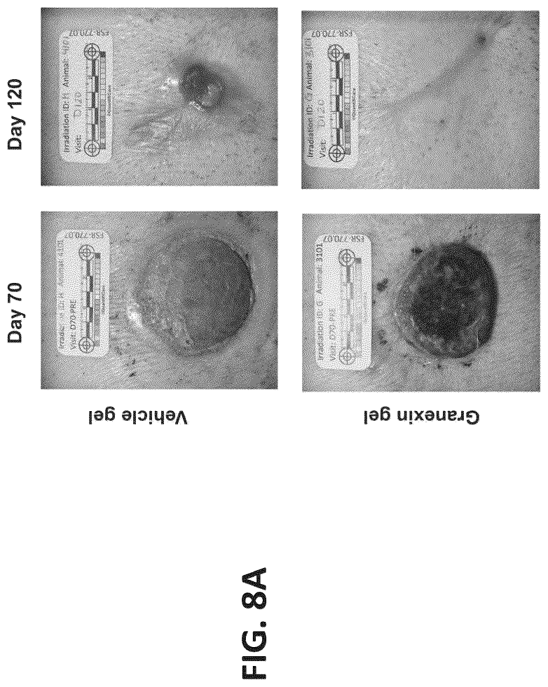

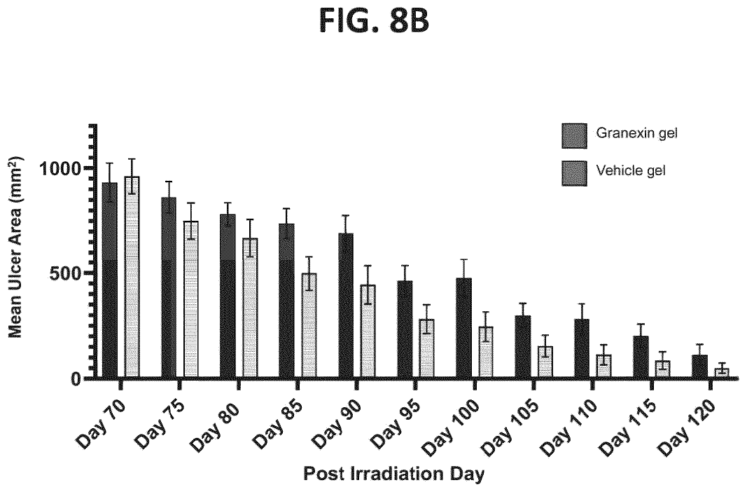

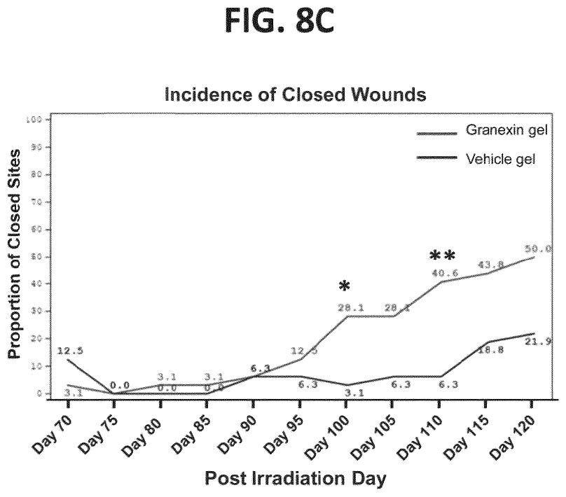

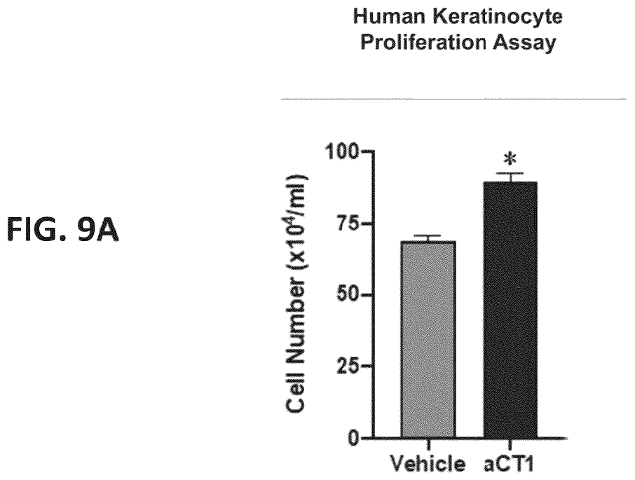

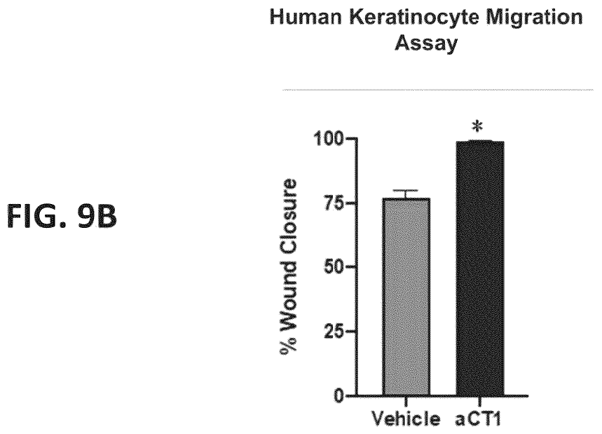

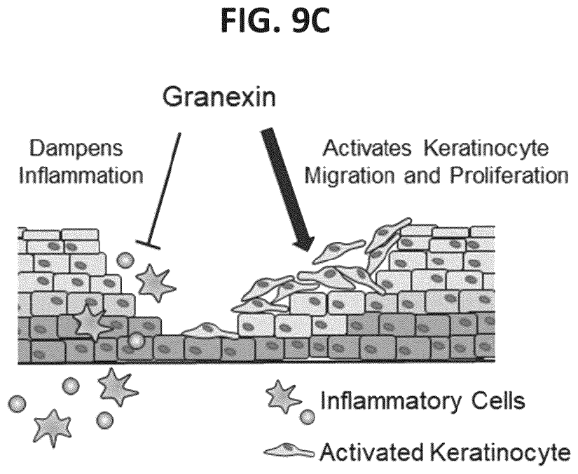

As shown in FIGS. 8A-8C, treatment with Granexin® gel on skin sites irradiated with 45 Gy accelerated wound re-epithelialization and increased incidence of closure of CRI wounds. As such, aCT1 (SEQ ID NO: 91) in Granexin® gel efficacy has been demonstrated when treatment is initiated after onset of visible signs and symptoms. Moreover, as shown in FIGS. 9A-9C, aCT1 peptide accelerated keratinocyte migration and proliferation to promote cutaneous healing. Overall, these results indicated that aCT1 (e.g., Granexin® gel with aCT1 peptide) is an effective medical countermeasure for the treatment of radiation induced skin injuries, and, by extension, may translate to other cutaneous wound indications such as thermal burns and chemical injuries.

### SEQUENCES OF THE DISCLOSURE WITH SEQ ID NO IDENTIFIERS

SEQ ID NO: 1 (ACT2)
PSSRASSRASSRPRPDDLEI
SEQ ID NO: 2 (ACT 1)
RPRPDDLEI
SEQ ID NO: 3 (ACT 3)
RPRPDDLEV
SEQ ID NO: 4 (ACT 4)
RPRPDDVPV
SEQ ID NO: 5 (ACT 5)
KARSDDLSV
SEQ ID NO: 6
aga cct cgg cct gat gac ctg gag att
SEQ ID NO: 7 (Antp)
RQPKIWFPNRRKPWKK
SEQ ID NO: 8 (Antp/ACT 2)
RQPKIWFPNRRKPWKKPSSRASSRASSRPRPDDLEI
SEQ ID NO: 9 (Antp/ACT 1)
RQPKIWFPNRRKPWKKRPRPDDLEI
SEQ ID NO: 10 (Antp/ACT 3)
RQPKIWFPNRRKPWKKRPRPDDLEV
SEQ ID NO: 11 (Antp/ACT4)
RQPKIWFPNRRKPWKKRPRPDDVPV
SEQ ID NO: 12 (Antp/ACT 5)
RQPKIWFPNRRKPWKKKARSDDLSV
SEQ ID NO: 13 (encodes polypeptide of SEQ ID NO 9)
cgg cag ccc aag atc tgg ttc ccc aac cgg aag ccc tgg aag cgg ccc ggc ccg acg acc tgg aga tc
SEQ ID NO: 14 (HIV-Tat)
GRKKRRQRPPQ
SEQ ID NO: 15 (Penetratin)
RQIKIWFQNRRMKWKK
SEQ ID NO: 16 (Antp-3A)
RQIAIWFQNRRMKWAA
SEQ ID NO: 17 (Tat)
RKKRRQRRR
SEQ ID NO: 18 (Buforin II)
TRSSRAGLQFPVGRVHRLLRK
SEQ ID NO: 19 (Transportan)
GWTLNSAGYLLGKINKALAALAKKIL
SEQ ID NO: 20 (model amphipathic peptide)
KLALKLALKALKAALKLA
SEQ ID NO: 21 (K-FGF)
AAVALLPAVLLALLAP
SEQ ID NO: 22 (Ku70)
VPMLK-PMLKE
SEQ ID NO: 23 (Prion)
MANLGYWLLALFVTMWTDVGLCKKRPKP
SEQ ID NO: 24 (pVEC)
LLIILRRRIRKQAHAHSK
SEQ ID NO: 25 (Pep-1)
KETWWETWWTEWSQPKKKRKV
SEQ ID NO: 26 (SynB1)
RGGRLSYSRRRFSTSTGR
SEQ ID NO: 27 (Pep-7)
SDLWEMMMVSLACQY
SEQ ID NO: 28 (HN-1)
TSPLNIHNGQKL
SEQ ID NO: 29 (Chick alpha Cx43 ACT)
PSRASSRASSRPRPDDLEI
SEQ ID NO: 30 (Human alpha Cx45)
GSNKSTASSKSPDPKNSVWI
SEQ ID NO: 31 (Chick alpha Cx45)
GSNKSSASSKSGDGKNSVWI
SEQ ID: 32 (Human alpha Cx46)
GRASKASRASSGRARPEDLAI
SEQ ID: 33 (Human alpha Cx46.6)
GSASSRDGKTVWI
SEQ ID NO: 34 (Chimp alpha Cx36)
PRVSVPNFGRTQSSDSAYV
SEQ ID NO: 35 (Chick alpha Cx36)
PRMSMPNFGRTQSSDSAYV
SEQ ID NO: 36 (Human alpha Cx47)
PRAGSEKGSASSRDGKTTVWI
SEQ ID NO: 37 (Human alpha Cx40)
GYHSDKRRLSKASSKARSDDLSV
SEQ ID NO: 38 (Human alpha Cx50)
PLSRLSKASSRARSDDLTV
SEQ ID NO: 39 (Human alpha Cx59)
PNHVVSLINNLIGRRVPTDLQI
SEQ ID NO: 40 (Rat alpha Cx33)
PSCVSSSAVLTTICSSDQVVPVGLSSFYM
SEQ ID NO: 41 (Sheep alpha Cx44)
GRSSKASKSSGGRARAADLAI
SEQ ID NO: 42 (Human beta Cx26)
LCYLLIRYCSGKSKKPV
SEQ ID: 43 (Human alpha Cx37)
GQKPPSRPSSSASKKQ*YV
SEQ ID 44: (conservative Cx43 variant)
SSRASSRASSRPRPDDLEV
SEQ ID 45: (conservative Cx43 variant)
RPKPDDLEI,
SEQ ID 46: (conservative Cx43 variant)
SSRASSRASSRPKPDDLEI,
SEQ ID 47: (conservative Cx43 variant)
RPKPDDLDI
SEQ ID 48: (conservative Cx43 variant)
SSRASSRASSRRPDDLDI
SEQ ID 49: (conservative Cx43 variant)
SSRASTRASSRPRPDDLEI
SEQ ID 50: (conservative Cx43 variant)
RPRPEDLEI
SEQ ID 51: (conservative Cx43 variant)
SSRASSRASSRPRPEDLEI,
SEQ ID 52: (conservative Cx45 variant)
GDGKNSVWV
SEQ ID 53: (conservative Cx45 variant)
SKAGSNKSTASSKSGDGKNSVWV
SEQ ID 54: (conservative Cx37 variant)
GQKPPSRPSSSASKKLYV
SEQ ID NO: 55 (non-active control peptide)
RQPKIWFPNRRKPWKIELDDPRPR
SEQ ID NO: 56 (HIV-Tat/ACT 1)
GRKKRRQRPPQRPRPDDLEI
SEQ ID NO: 57 (Penetratin/ACT 1)
RQIKIWFQNRRMKWKKRPRPDDLEI
SEQ ID NO: 58 (Antp-3A/ACT 1)
RQIAIWFQNRRMKWAARPRPDDLEI
SEQ ID NO: 59 (Tat/ACT 1)
RKKRRQRRRRPRPDDLEI
SEQ ID NO: 60 (Buforin II/ACT 1)
TRSSRAGLQFPVGRVHRLLRKRPRPDDLEI
SEQ ID NO: 61 (Transportan/ACT 1)
GWTLNSAGYLLGKINKALAALAKKILRPRPDDLEI
SEQ ID NO: 62 (MAP/ACT 1)
KLALKLALKALKAALKLARPRPDDLEI
SEQ ID NO: 63 (K-FGF/ACT 1)
AAVALLPAVLLALLAPRPRPDDLEI
SEQ ID NO: 64 (Ku70/ACT 1)
VPMLKPMLKERPRPDDLEI
SEQ ID NO: 65 (Prion/ACT 1)
MANLGYWLLALFVTMWTDVGLCKKRPKPRPRPDDLEI
SEQ ID NO: 66 (pVEC/ACT 1)
LLIILRRRIRKQAHAHSKRPRPDDLEI
SEQ ID NO: 67 (Pep-1/ACT 1)
KETWWETWWTEWSQPKKKRKVRPRPDDLEI
SEQ ID NO: 68 (SynB1/ACT 1)
RGGRLSYSRRRFSTSTGRRPRPDDLEI
SEQ ID NO: 69 (Pep-7/ACT 1)
SDLWEMMMVSLACQYRPRPDDLEI
SEQ ID NO: 70 (HN-1/ACT 1)
TSPLNIHNGQKLRPRPDDLEI
SEQ ID NO: 72 (20 to 120 residues flanking amino acid 363 of human Cx43)
KGKSDPYHATSGALSPAKDCGSQKYAYFNGCSSPTAPLSPMSPPGYKLVT
GDRNNSSCRNYNKQASEQNWANYSAEQNRMGQAGSTISNSHAQPFDFPDD
NQNSKKLAAGHELQPLAIVD
SEQ ID NO: 73 (20 to 120 residues flanking amino acid 362 of chick Cx43)
KTDPYSHSGTMSPSKDCGSPKYAYYNGCSSPTAPLSPMSPPGYKLVTGDR
NNSSCRNYNKQASEQNWANYSAEQNRMGQAGSTISNSHAQPFDFADEHQN
TKKLASGHELQPLTIVDORP
SEQ ID NO: 74 (20 to 120 residues flanking amino acid 377 of human Cx45)
LGFGTIRDSLNSKRRELEDPGAYNYPFTWNTPSAPPGYNIAVKPDQIQYT
ELSNAKIAYKQNKANTAQEQQYGSHEENLPADLEALQREIRMAQERLDLA
VQAYSHONNPHGPREKKAKV
SEQ ID NO: 75 (20 to 120 residues flanking amino acid 375 of chick Cx45)
GFGTIRDTLNNKRKELEDSGTYNYPFTWNTPSAPPGYNIAVKPDQMQYTE
LSNAKMAYKONKANIAQEQQYGSNEENIPADLENLQREIKVAQERLDMAI
QAYNNQNNPGSSSREKKSKA.
SEQ ID NO: 76 (20 to 120 residues flanking amino acid 313 of human Cx37)
PYLVDCFVSRPTEKTIFIIFMLVVGLISLVLNLLELVHLLCRCLSRGMRA
RQGQDAPPTQGTSSDPYTDQVFFYLPVGQGPSSPPCPTYNGLSSSEQNWA
NLTTEERLASSRPPLFLDPP
SEQ ID NO: 77 (20 to 120 residues flanking amino acid 258 of rat Cx33)
CGSKEHGNRKMRGRLLLTYMASIFFKSVFEVAFLLIQWYLYGFTLSAVYI
CEQSPCPHRVDCFLSRPTEKTIFILFMLVVSMVSFVLNVIELFYVLFKAI
KNHLGNEKEEVYCNPVELQK.
SEQ ID NO: 78 (enhanced green fluorescent protein)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYK
SEQ ID NO: 78 (ACT 2)
CCCTCCTCCCGGGCCTCCTCCCGGGCCTCCTCCCGGCCCCGGCCCGACG
ACCTGGAGATC
SEQ ID NO: 79 (ACT 1)
CGGCCCCGGCCCGACGACCTGGAGATC
SEQ ID NO: 80 (ACT 3)
CGGCCCCGGCCCGACGACCTGGAGGTG
SEQ ID NO: 81 (ACT 4)
CGGCCCCGGCCCGACGACGTGCCCGTG
SEQ ID NO: 82 (ACT 5)
AAGGCCCGGTCCGACGACCTGTCCGTG
SEQ ID NO: 83 (Antp)
CGGCAGCCCAAGATCTGGTTCCCCAACCGGCGGAAGCCCTGGAAGAAG
SEQ ID NO: 84 (Antp/ACT 2)
CGGCAGCCCAAGATCTGGTTCCCCAACCGGCGGAAGCCCTGGAAGAAGCC
CTCCTCCCGGGCCTCCTCCCGGGCCTCCTCCCGGCCCCGGCCCGACGACC
TGGAGATC
SEQ ID NO: 85 (Antp/ACT 1)
CGGCAGCCCAAGATCTGGTTCCCCAACCGGCGGAAGCCCTGGAAGAAGCG
GCCCCGGCCCGACGACCTGGAGATC
SEQ ID NO: 86 (Antp/ACT 3)
CGGCAGCCCAAGATCTGGTTCCCCAACCGGCGGAAGCCCTGGAAGAAGCG
GCCCCGGCCCGACGACCTGGAGGTG
SEQ ID NO: 87 (Antp/ACT 4)
CGGCAGCCCAAGATCTGGTTCCCCAACCGGCGGAAGCCCTGGAAGAAGCG
GCCCCGGCCCGACGACGTGCCCGTG
SEQ ID NO: 88 (Antp/ACT 5)
CGGCAGCCCAAGATCTGGTTCCCCAACCGGCGGAAGCCCTGGAAGAAGAA
GGCCCGGTCCGACGACCTGTCCGTG
SEQ ID NO: 89 (Zebrafish alpha Cx43)
PCSRASSRMSSRARPDDLDV
SEQ ID NO: 90 (Chick alpha Cx36)
PRVSVPNFGRTQSSDSAYV
SEQ ID NO: 91 (Peptide 328967 (ACT 1))
Biotin-Ahx-Arg-Gln-Pro-Lys-Ile-Trp-Phe-Pro-Asn-Arg-Arg-Lys-Pro-Trp-Lys-Lys-Arg-Pro-
Arg-Pro-Asp-Asp-Leu-Glu-Ile-OH

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed:

1. A method of treating a radiation induced skin injury on a subject, comprising:
(a) debriding a radiation induced skin injury on a subject exposed to a source of radiation; and
(b) administering to the subject a composition comprising at least one alpha connexin polypeptide in a dosing regimen effective for the treatment of the radiation induced skin injury, and wherein step (a) is repeated at defined intervals during the dosing regimen effective for the treatment of the radiation induced skin injury.

2. The method of claim 1, wherein the dosing regimen effective for the treatment of the radiation induced skin injury is daily for a plurality of days.

3. The method of claim 2, wherein the plurality of days is 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115 or 120 days.

4. The method of any one of the above claims, wherein the defined intervals is daily, every 2 days, every 3 days, every 4 days, every 5 days or every 6 days.

5. The method of any one of the above claims, wherein the composition is administered to the subject at a dose of from about 10 μM to about 2000 μM.

6. The method of claim 5, wherein the composition is administered to the subject at a dose of from about 100 μM to about 200 μM.

7. The method of any one of claims 1-4, wherein the composition is administered to the subject at a dose of about 1 mg/kg to about 50 mg/kg.

8. The method of any one of the above claims, wherein the composition is administered topically.

9. The method of claim 8, wherein the composition further comprises hydroxyethylcellulose gel.

10. The method of claim 9, wherein the hydroxyethylcellulose gel is present at a concentration of about 1.25% (w/w).

11. The method of any one of the above claims, wherein the at least one alpha connexin polypeptide comprises the carboxy terminal-most 4 to 30 contiguous amino acids of an alpha Connexin, or a conservative variant thereof.

12. The method of claim 11, wherein the alpha connexin polypeptide is connexin 37,connexin 40, connexin 43, or connexin 45.

13. The method of any one of the above claims, wherein the at least one alpha connexin polypeptide is linked at its amino terminus to a cellular internalization transporter.

14. The method of claim 13, wherein the cellular internalization transporter is an antennapedia sequence.

15. The method of any one of the above claims, wherein the at least alpha connexin polypeptide comprises the amino acid sequence of SEQ ID NO: 9.

16. The method of any one of the above claims, wherein the at least alpha connexin polypeptide comprises the amino acid sequence of SEQ ID NO: 91.

17. The method of any one of the above claims, wherein step (b) is performed after the onset of erythema, desquamation, ulceration or any combination thereof.

18. The method of any one of the above claims, wherein step (b) is performed after the onset of erythema.

19. The method of any one of the above claims, wherein the radiation induced skin injury is selected from the group consisting of cutaneous radiation injury (CRI), radiation burns, and radiation dermatitis.

20. The method of any one of the above claims, wherein the source of radiation is selected from the group consisting of a medical intervention, a diagnostic machine, the sun, a tanning bed, weapons of mass destruction (WMD), nuclear explosions, and dirty bombs.

21. The method of any one of the above claims, wherein the method reduces the time to 50% wound closure of the radiation induced skin injury as compared to standard of care treatment for wound healing of the radiation induced skin injury.

22. The method of any one of the above claims, wherein the method reduces the time to 100% wound closure of the radiation induced skin injury as compared to standard of care treatment for wound healing of the radiation induced skin injury.

23. The method of any one of the above claims, wherein the method increases the incidence or frequency of 100% complete wound closure of the radiation induced skin injury as compared to standard of care treatment for wound healing of the radiation induced skin injury.

24. The method of any one of the above claims, wherein the method increases the average percent of wound closure of the radiation induced skin injury as compared to standard of care treatment for wound healing of the radiation induced skin injury.

25. The method of any one of the above claims, wherein the method accelerates wound re-epithelialization of the radiation induced skin injury by at least 5%, 10%, 15%, 20%, 25%, 30%, 35%, 40%, 45%, 50%, 55%, 60%, 65%, 70%, 75%, 80%, 85%, 90%, 95% 99% or 100% as compared to standard of care treatment for wound healing of the radiation induced skin injury.

26. The method of any one of the above claims, wherein the debriding performed in step (a) comprises one or more types of debridement selected from the group consisting of autolytic, surgical, mechanical, irrigation, biological, enzymatic and any combination thereof.

27. The method of claim 26, wherein the type of debridement performed in step (a) is determined following evaluation by a caregiver.

28. The method of claim 27, wherein the caregiver is a certified medical professional.


SEQUENCE LISTING (91 sequences)

SEQ ID NO: 1 - protein, 20 aa, synthetic construct
PSSRASSRASSRPRPDDLEI

SEQ ID NO: 2 - protein, 9 aa, synthetic construct
RPRPDDLEI

SEQ ID NO: 3 - protein, 9 aa, synthetic construct
RPRPDDLEV

SEQ ID NO: 4 - protein, 9 aa, synthetic construct
RPRPDDVPV

SEQ ID NO: 5 - protein, 9 aa, synthetic construct
KARSDDLSV

SEQ ID NO: 6 - DNA, 27 bp, synthetic construct
  source 1..27: mol_type = other DNA
agacctcggcctgatgacctggagatt

SEQ ID NO: 7 - protein, 16 aa, synthetic construct
RQPKIWFPNRRKPWKK

SEQ ID NO: 8 - protein, 36 aa, synthetic construct
RQPKIWFPNRRKPWKKPSSRASSRASSRPRPDDLEI

SEQ ID NO: 9 - protein, 25 aa, synthetic construct
RQPKIWFPNRRKPWKKRPRPDDLEI

SEQ ID NO: 10 - protein, 25 aa, synthetic construct
RQPKIWFPNRRKPWKKRPRPDDLEV

SEQ ID NO: 11 - protein, 25 aa, synthetic construct
RQPKIWFPNRRKPWKKRPRPDDVPV

SEQ ID NO: 12 - protein, 25 aa, synthetic construct
RQPKIWFPNRRKPWKKKARSDDLSV

SEQ ID NO: 13 - DNA, 74 bp, synthetic construct
  source 1..74: mol_type = other DNA
cggcagcccaagatctggttccccaaccggcggaagccctggaagaagcggcccggcccgacgacctggagatc

SEQ ID NO: 14 - protein, 11 aa, synthetic construct
GRKKRRQRPPQ

SEQ ID NO: 15 - protein, 16 aa, synthetic construct
RQIKIWFQNRRMKWKK

SEQ ID NO: 16 - protein, 16 aa, synthetic construct
RQIAIWFQNRRMKWAA

SEQ ID NO: 17 - protein, 9 aa, synthetic construct
RKKRRQRRR

SEQ ID NO: 18 - protein, 21 aa, synthetic construct
TRSSRAGLQFPVGRVHRLLRK

SEQ ID NO: 19 - protein, 26 aa, synthetic construct
GWTLNSAGYLLGKINKALAALAKKIL

SEQ ID NO: 20 - protein, 18 aa, synthetic construct
KLALKLALKALKAALKLA

SEQ ID NO: 21 - protein, 16 aa, synthetic construct
AAVALLPAVLLALLAP

SEQ ID NO: 22 - protein, 10 aa, synthetic construct
VPMLKPMLKE

SEQ ID NO: 23 - protein, 28 aa, synthetic construct
MANLGYWLLALFVTMWTDVGLCKKRPKP

SEQ ID NO: 24 - protein, 18 aa, synthetic construct
LLIILRRRIRKQAHAHSK

SEQ ID NO: 25 - protein, 21 aa, synthetic construct
KETWWETWWTEWSQPKKKRKV

SEQ ID NO: 26 - protein, 18 aa, synthetic construct
RGGRLSYSRRRFSTSTGR

SEQ ID NO: 27 - protein, 15 aa, synthetic construct
SDLWEMMMVSLACQY

SEQ ID NO: 28 - protein, 12 aa, synthetic construct
TSPLNIHNGQKL

SEQ ID NO: 29 - protein, 19 aa, synthetic construct
PSRASSRASSRPRPDDLEI

SEQ ID NO: 30 - protein, 20 aa, synthetic construct
GSNKSTASSKSPDPKNSVWI

SEQ ID NO: 31 - protein, 20 aa, synthetic construct
GSNKSSASSKSGDGKNSVWI

SEQ ID NO: 32 - protein, 21 aa, synthetic construct
GRASKASRASSGRARPEDLAI

SEQ ID NO: 33 - protein, 13 aa, synthetic construct
GSASSRDGKTVWI

SEQ ID NO: 34 - protein, 19 aa, synthetic construct
PRVSVPNFGRTQSSDSAYV

SEQ ID NO: 35 - protein, 19 aa, synthetic construct
PRMSMPNFGRTQSSDSAYV

SEQ ID NO: 36 - protein, 21 aa, synthetic construct
PRAGSEKGSASSRDGKTTVWI

SEQ ID NO: 37 - protein, 23 aa, synthetic construct
GYHSDKRRLSKASSKARSDDLSV

SEQ ID NO: 38 - protein, 19 aa, synthetic construct
PLSRLSKASSRARSDDLTV

SEQ ID NO: 39 - protein, 22 aa, synthetic construct
PNHVVSLTNNLIGRRVPTDLQI

SEQ ID NO: 40 - protein, 29 aa, synthetic construct
PSCVSSSAVLTTICSSDQVVPVGLSSFYM

SEQ ID NO: 41 - protein, 21 aa, synthetic construct
GRSSKASKSSGGRARAADLAI

SEQ ID NO: 42 - protein, 17 aa, synthetic construct
LCYLLIRYCSGKSKKPV

SEQ ID NO: 43 - protein, 18 aa, synthetic construct
GQKPPSRPSSSASKKQYV

SEQ ID NO: 44 - protein, 19 aa, synthetic construct
SSRASSRASSRPRPDDLEV

SEQ ID NO: 45 - protein, 9 aa, synthetic construct
RPKPDDLEI

SEQ ID NO: 46 - protein, 19 aa, synthetic construct
SSRASSRASSRPKPDDLEI

SEQ ID NO: 47 - protein, 9 aa, synthetic construct
RPKPDDLDI

SEQ ID NO: 48 - protein, 19 aa, synthetic construct
SSRASSRASSRPRPDDLDI

SEQ ID NO: 49 - protein, 19 aa, synthetic construct
SSRASTRASSRPRPDDLEI

SEQ ID NO: 50 - protein, 9 aa, synthetic construct
RPRPEDLEI

SEQ ID NO: 51 - protein, 19 aa, synthetic construct
SSRASSRASSRPRPEDLEI

SEQ ID NO: 52 - protein, 9 aa, synthetic construct
GDGKNSVWV

SEQ ID NO: 53 - protein, 23 aa, synthetic construct
SKAGSNKSTASSKSGDGKNSVWV

SEQ ID NO: 54 - protein, 18 aa, synthetic construct
GQKPPSRPSSSASKKLYV

SEQ ID NO: 55 - protein, 24 aa, synthetic construct
RQPKIWFPNRRKPWKIELDDPRPR

SEQ ID NO: 56 - protein, 20 aa, synthetic construct
GRKKRRQRPPQRPRPDDLEI

SEQ ID NO: 57 - protein, 25 aa, synthetic construct
RQIKIWFQNRRMKWKKRPRPDDLEI

SEQ ID NO: 58 - protein, 25 aa, synthetic construct
RQIAIWFQNRRMKWAARPRPDDLEI

SEQ ID NO: 59 - protein, 18 aa, synthetic construct
RKKRRQRRRRPRPDDLEI

SEQ ID NO: 60 - protein, 30 aa, synthetic construct
TRSSRAGLQFPVGRVHRLLRKRPRPDDLEI

SEQ ID NO: 61 - protein, 35 aa, synthetic construct
GWTLNSAGYLLGKINKALAALAKKILRPRPDDLEI

SEQ ID NO: 62 - protein, 27 aa, synthetic construct
KLALKLALKALKAALKLARPRPDDLEI

SEQ ID NO: 63 - protein, 25 aa, synthetic construct
AAVALLPAVLLALLAPRPRPDDLEI

SEQ ID NO: 64 - protein, 19 aa, synthetic construct
VPMLKPMLKERPRPDDLEI

SEQ ID NO: 65 - protein, 37 aa, synthetic construct
MANLGYWLLALFVTMWTDVGLCKKRPKPRPRPDDLEI

SEQ ID NO: 66 - protein, 27 aa, synthetic construct
LLIILRRRIRKQAHAHSKRPRPDDLEI

SEQ ID NO: 67 - protein, 30 aa, synthetic construct
KETWWETWWTEWSQPKKKRKVRPRPDDLEI

SEQ ID NO: 68 - protein, 27 aa, synthetic construct
RGGRLSYSRRRFSTSTGRRPRPDDLEI

SEQ ID NO: 69 - protein, 24 aa, synthetic construct
SDLWEMMMVSLACQYRPRPDDLEI

SEQ ID NO: 70 - protein, 21 aa, synthetic construct
TSPLNIHNGQKLRPRPDDLEI

SEQ ID NO: 71 - protein, 120 aa, synthetic construct
KGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVD

SEQ ID NO: 72 - protein, 120 aa, synthetic construct
KTDPYSHSGTMSPSKDCGSPKYAYYNGCSSPTAPLSPMSPPGYKLVTGDRNNSSCRNYNKQASEQNWANYSAEQNRMGQAGSTISNSHAQPFDFADEHQNTKKLASGHELQPLTIVDQRP

SEQ ID NO: 73 - protein, 120 aa, synthetic construct
LGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKV

SEQ ID NO: 74 - protein, 120 aa, synthetic construct
GFGTIRDTLNNKRKELEDSGTYNYPFTWNTPSAPPGYNIAVKPDQMQYTELSNAKMAYKQNKANIAQEQQYGSNEENIPADLENLQREIKVAQERLDMAIQAYNNQNNPGSSSREKKSKA

SEQ ID NO: 75 - protein, 120 aa, synthetic construct
PYLVDCFVSRPTEKTIFIIFMLVVGLISLVLNLLELVHLLCRCLSRGMRARQGQDAPPTQGTSSDPYTDQVFFYLPVGQGPSSPPCPTYNGLSSSEQNWANLTTEERLASSRPPLFLDPP

SEQ ID NO: 76 - protein, 120 aa, synthetic construct
CGSKEHGNRKMRGRLLLTYMASIFFKSVFEVAFLLIQWYLYGFTLSAVYICEQSPCPHRVDCFLSRPTEKTIFILFMLVVSMVSFVLNVIELFYVLFKAIKNHLGNEKEEVYCNPVELQK

SEQ ID NO: 77 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 78 - DNA, 60 bp, synthetic construct
  source 1..60: mol_type = other DNA
ccctcctcccgggcctcctcccgggcctcctcccggccccggcccgacgacctggagatc

SEQ ID NO: 79 - DNA, 27 bp, synthetic construct
  source 1..27: mol_type = other DNA
cggccccggcccgacgacctggagatc

SEQ ID NO: 80 - DNA, 27 bp, synthetic construct
  source 1..27: mol_type = other DNA
cggccccggcccgacgacctggaggtg

SEQ ID NO: 81 - DNA, 27 bp, synthetic construct
  source 1..27: mol_type = other DNA
cggccccggcccgacgacgtgcccgtg

SEQ ID NO: 82 - DNA, 27 bp, synthetic construct
  source 1..27: mol_type = other DNA
aaggcccggtccgacgacctgtccgtg

SEQ ID NO: 83 - DNA, 48 bp, synthetic construct
  source 1..48: mol_type = other DNA
cggcagcccaagatctggttccccaaccggcggaagccctggaagaag

SEQ ID NO: 84 - DNA, 108 bp, synthetic construct
  source 1..108: mol_type = other DNA
cggcagcccaagatctggttccccaaccggcggaagccctggaagaagccctcctcccgggcctcctcccgggcctcctcccggccccggcccgacgacctggagatc

SEQ ID NO: 85 - DNA, 75 bp, synthetic construct
  source 1..75: mol_type = other DNA
cggcagcccaagatctggttccccaaccggcggaagccctggaagaagcggccccggcccgacgacctggagatc

SEQ ID NO: 86 - DNA, 75 bp, synthetic construct
  source 1..75: mol_type = other DNA
cggcagcccaagatctggttccccaaccggcggaagccctggaagaagcggccccggcccgacgacctggaggtg

SEQ ID NO: 87 - DNA, 75 bp, synthetic construct
  source 1..75: mol_type = other DNA
cggcagcccaagatctggttccccaaccggcggaagccctggaagaagcggccccggcccgacgacgtgcccgtg

SEQ ID NO: 88 - DNA, 75 bp, synthetic construct
  source 1..75: mol_type = other DNA
cggcagcccaagatctggttccccaaccggcggaagccctggaagaagaaggcccggtccgacgacctgtccgtg

SEQ ID NO: 89 - protein, 20 aa, synthetic construct
PCSRASSRMSSRARPDDLDV

SEQ ID NO: 90 - protein, 19 aa, synthetic construct
PRVSVPNFGRTQSSDSAYV

SEQ ID NO: 91 - protein, 26 aa, synthetic construct
  SITE 1: note = N-terminus is biotinylated
  SITE 1: note = Nle
XRQPKIWFPNRRKPWKKRPRPDDLEI